(19) United States
(12) Patent Application Publication
(10) Pub. No.: US 2010/0190198 A1
(43) Pub. Date: Jul. 29, 2010

(54) ASYMMETRIC SYSTEMS

(76) Inventors: Osman Kibar, San Diego, CA (US); Mirianas Chachisvilis, San Diego, CA (US); Eugene Tu, San Diego, CA (US)

Correspondence Address: FISH & RICHARDSON PC, P.O. BOX 1022, MINNEAPOLIS, MN, 55440-1022, US

(21) Appl. No.: 12/543,190

(22) Filed: Aug. 18, 2009

Related U.S. Application Data
(60) Provisional application No. 61/090,028, filed on Aug. 19, 2008.
(60) Provisional application No. 61/171,645, filed on Apr. 22, 2009.
(60) Provisional application No. 61/172,838, filed on Apr. 27, 2009.
(60) Provisional application No. 61/172,959, filed on Apr. 27, 2009.
(60) Provisional application No. 61/179,233, filed on May 18, 2009.

(51) Int. Cl. F03G 7/00; F03G 7/08; H02N 2/00; F02B 63/04; C12P 1/00; C01B 39/00; C08F 2/00; D01F 9/12; B01J 19/00; C07C 4/02; C07C 31/02; C07H 1/00; C07C 2/00; C09K 3/00; B01J 23/00; B01D 53/04; C07K 2/00; C12Q 1/02; C12N 1/00

(52) U.S. Cl. 435/29; 60/641.1; 290/1 R; 310/339; 435/41; 423/700; 528/480; 423/447.1; 422/129; 208/106; 568/840; 536/124; 585/500; 252/182.11; 502/100; 423/230; 530/350; 435/243; 708/405; 708/404; 977/742; 708/400; 708/403

(57) ABSTRACT

Among other things, a combination comprises interaction with a system that has a perturbation. In such perturbed system, a non-directional input is applied to a first variable of the system. Based on an asymmetry of the perturbed system, a directional effect is achieved in a second variable of the system, the first and second variables comprising a conjugate pair of variables. At least one of the following pertains: the interaction occurs other than by an apparatus and other than in a way that actually achieves the directional effect, or the conjugate pair is other than position and momentum, or the input or the asymmetry are in a dimension other than spatial coordinates, or the directional effect is other than translational motion and other than rotary motion.

This application is entitled to the benefit of the priority of U.S. provisional application Ser. 61/090,028, filed Aug. 19, 2008, U.S. provisional application Ser. 61/171,645, filed Apr. 22, 2009, U.S. provisional application Ser. 61/172,838, filed Apr. 27, 2009, U.S. provisional application Ser. 61/172,959, filed Apr. 27, 2009, and U.S. provisional application Ser. 61/179,233, filed May 18, 2009, all of which are incorporated here in their entireties by reference.

### BACKGROUND

This description relates to asymmetric systems.

In a Brownian ratchet, for example, a force that is not directional in space, e.g., its average over space is zero, generates a directional motion of particles in a system.

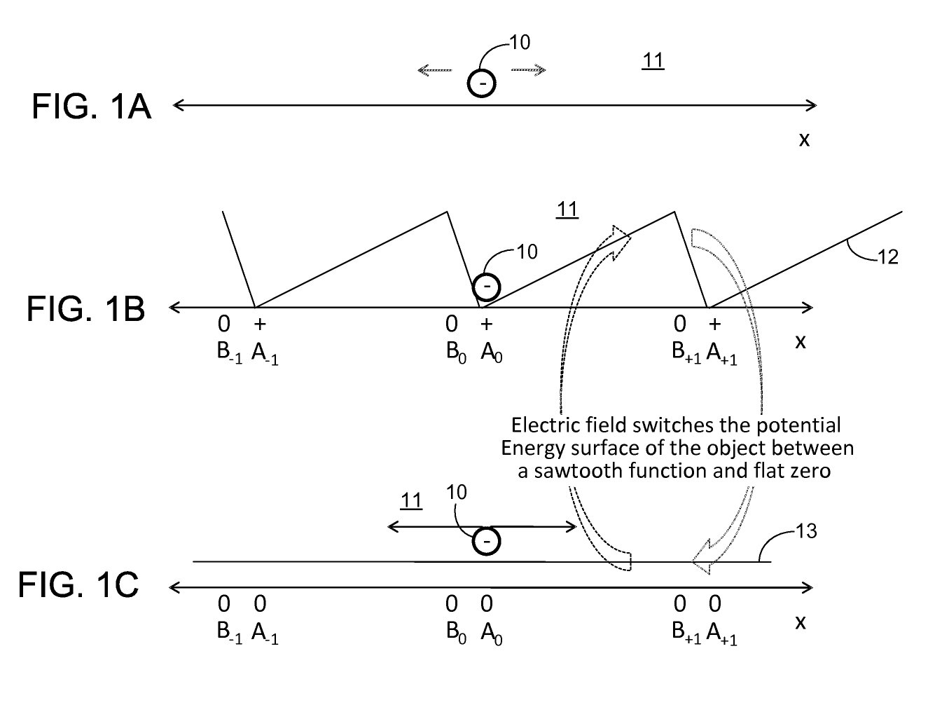

In FIGS. 1A, 1B, and 1C, for example, assume that a negatively charged particle 10 (e.g., a molecule in a liquid) is in thermal equilibrium with its environment 11. In a one-dimensional system, the molecule can move only along the x-axis, i.e., to the right or to the left in FIG. 1A.

An external electric field (constant in time) is applied to the system to create a saw-tooth-shaped energy profile 12 to which the molecule is subjected as shown in FIG. 1B. To achieve this energy profile in the case of a negatively charged molecule, a positive voltage is applied at each point along the x-dimension that corresponds to a minimum in energy (e.g., A₀), and a zero voltage is applied to each point that corresponds to a maximum in energy (e.g., B₀). When this field is being applied, the molecule will move towards a point of minimum energy, e.g., A₀, if it is not already at such a point. In the example shown, the A points and the B points are located periodically along the axis and the distance between each A point and the next adjacent B point to its left is less than the distance from that A point to the next adjacent B point to its right.

When the field is turned off, a molecule that is at one of the points of minimum energy, say A₀, is subjected to an energy profile 13 that is flat as shown in FIG. 1C. With no energy barriers to either side along the x-axis, the molecule will experience Brownian motion and diffuse to either side of A₀ with equal probability. The molecule's diffusion away from point A₀ constitutes a perturbation of the system.

Assume that the field is kept off until the probability of the molecule diffusing at least a distance (B₀−A₀) is significant, but the probability of the molecule diffusing at least a distance (B₊₁−A₀) is still low. When the electric field is turned back on to restore the energy profile of FIG. 1B, if the molecule had diffused to a point to the left of B₀, it will move (slide down the energy profile) towards A₋₁ under the influence of the electric field. On the other hand, if the molecule had diffused to the right beyond B₊₁, the restored field would cause it to move (slide down the energy profile) towards A₊₁.

However, because the distance (B₀−A₀) is shorter than the distance (B₊₁−A₀), at the instant the field is turned on, the probability of the particle being to the left of B₀ will be greater than of being to the right of B₊₁ because the diffusion is not statistically preferential in either direction. In other words the probability is higher that the molecule will have taken a step to the left than to the right (by a step we mean a distance that puts it beyond the next energy peak along the direction in which it diffuses).

When this cycle (of turning the field on and off) is repeated many times, the molecule will, on average, have taken more steps to the left than to the right, and therefore have experienced directional motion to the left, even though the applied force (induced by the electric field) is not directional when averaged over space.

Such directional motion can overcome even an opposing load (e.g., a force tending to push the molecule to the right). In that case, the applied non-directional force of the electric field would be doing directional work, pushing the molecule to the left despite the opposing force.

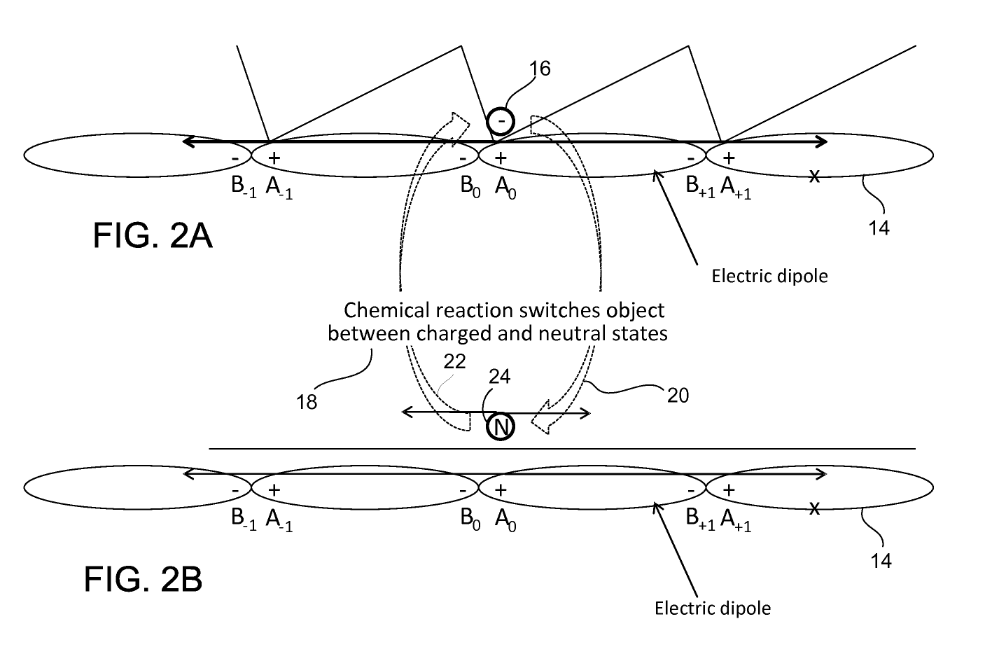

Another known type of Brownian ratchet is a flashing ratchet [Astumian R D and Bier M, “Fluctuation driven ratchets—molecular motors”, Phys. Rev. Lett. 72 1766-9, 1994]. In a flashing ratchet, instead of applying an asymmetric voltage profile externally to a system (as in FIG. 1B), a molecular track of electric dipoles 14 is arranged in a row (FIGS. 2A and 2B) along the x-axis. The molecule moves along the linear track. The spacing of the successive negative and positive charges (labeled B and A) along the track is asymmetric (e.g., the distance from one negative charge to the adjacent positive charge in one direction along the track is different from the distance between that positive charge and the next negative charge in the same direction along the track). Furthermore, the molecule 16 is not permanently charged. Instead, a chemical reaction 18 (e.g., an enzymatic conversion) switches the molecule back and forth 20, 22 between a charged state 16 and an electrically neutral state 24. And because the exact timing of such a chemical reaction is stochastic (as opposed to a deterministic voltage profile applied externally), the charge of the molecule (and thus, the potential energy profile that the molecule experiences from the track) “flashes”. Another example of a flashing ratchet uses an asymmetric molecular track arranged as a circle to generate rotary motion (Jiufu Lim, John E. Sader, and Paul Mulvaney, “Electrodynamic ratchet motor,” Physical Review E, 79, pp 030105-1-4, 2009).

Brownian ratchets have been applied in Brownian motors and Brownian pumps respectively to move particles directionally, e.g., against an opposing force, and to pump particles (e.g., ions) against a concentration and/or a voltage gradient. A review of Brownian ratchets can be found in various articles (e.g., Astumian and Derenyi, “Fluctuation driven transport and models of molecular motors and pumps”, European Biophysics Journal, vol 27, pp 474-489, 1998, and the references mentioned in that article).

Another ratchet mechanism is the Feynman ratchet [R. P. Feynman, R. B. Leighton, M. Sands, The Feynman Lectures on Physics (Addison-Wesley, Reading, Mass., 1966), vol. 1, chap. 46]. In this example, a ratchet and a pawl are in two thermally separated reservoirs at different temperatures. An asymmetry in the system—a difference in temperature between the two reservoirs—leads to a directional rotation of the ratchet mechanism (which can do work against a load).

There are also mechanical ratchets that achieve linear or rotary motion in one direction, while preventing motion in the opposite direction (Ref http://en.wikipedia.org/wiki/Ratchet_(device)).

### SUMMARY

In general, in an aspect, a combination comprises interaction with a system that has a perturbation. In such perturbed system, a non-directional input is applied to a first variable of the system. Based on an asymmetry of the perturbed system, a directional effect is achieved in a second variable of the system, the first and second variables comprising a conjugate pair of variables. At least one of the following pertains: the interaction occurs other than by an apparatus and other than in a way that actually achieves the directional effect, or the conjugate pair is other than position and momentum, or the input or the asymmetry is in a dimension other than spatial coordinates, or the directional effect is other than one-dimensional translational motion and other than one-dimensional rotary motion.

Implementations may include one or more of the following features. The interaction comprises causing the system to actually achieve the directional effect. The interaction includes an apparatus. The interaction comprises designing the system. The interaction comprises manipulating the system. The manipulating comprises analyzing the system. The manipulating comprises optimizing the system. The system is optimized for work done, for energy efficiency, for operation in a desired regime, or for a particular load.

The manipulation comprises implementing a function in the system. The function comprises filtering. The function comprises adaptive filtering. The function comprises compression. The function comprises de-compression. The function comprises sampling. The function comprises de-sampling. The function comprises feature extraction. The function comprises spectrum analysis. The function comprises storage. The function comprises modulation.

The interaction is based on signal processing. The signal processing comprises description of an aspect of the system. The signal processing comprises interpretation of an aspect of the system.

The signal processing comprises taking the transform of a variable. The transform comprises an integral transform. The integral transform comprises a Fourier transform. The integral transform comprises a Laplace transform. The integral transform comprises a wavelet transform. The integral transform comprises a Hilbert transform. The transform comprises a discrete transform. The discrete transform comprises a binomial transform. The discrete transform comprises a discrete Fourier transform. The discrete transform comprises a fast Fourier transform. The discrete transform comprises a Z-transform. The transform comprises a data-dependent transform. The transform comprises a transform other than an integral, discrete or data-dependent transform. The transform comprises a one-variable transform. The transform comprises a multi-variable transform.

The system comprises a physical system. The system comprises a chemical system. The system comprises a chemical reaction. The chemical reaction comprises an intermediate chemical reaction. The chemical reaction comprises a surface reaction. The chemical reaction comprises a bulk reaction. The chemical reaction comprises a membrane reaction. The chemical reaction comprises an organic reaction. The chemical reaction comprises an inorganic reaction. The chemical reaction comprises an enzymatic reaction. The chemical reaction comprises catalytic reaction. The chemical reaction comprises a non-catalytic reaction. The chemical reaction comprises a spontaneous reaction. The chemical reaction comprises a non-spontaneous reaction. The chemical reaction comprises an exothermic reaction. The chemical reaction comprises an endothermic reaction. The chemical reaction comprises a single chemical path. The chemical reaction comprises multiple possible chemical paths.

The system comprises a biological system. The system comprises a social system. The system comprises an economic system. The system comprises a combination of two or more of physical, chemical, biological, social and economic systems.

The conjugate pair of variables comprises position and momentum. The conjugate pair of variables comprises time and energy. The conjugate pair of variables comprises temperature and entropy. The conjugate pair of variables comprises pressure and volume. The conjugate pair of variables comprises electric field and polarizability. The conjugate pair of variables comprises magnetic field and magnetization. The conjugate pair of variables comprises stress and strain. The conjugate pair of variables comprises rotation angle and angular momentum. The conjugate pair of variables comprises chemical potential and particle number. The conjugate pair of variables comprises electric potential and electromotive force. The conjugate pair of variables comprises two orthogonal polarization vectors of an electromagnetic beam. The conjugate pair of variables comprises surface area and surface tension. There are two or more conjugate pairs of variables in the system.

The system comprises a feedback system. The system comprises a time-invariant system. The system comprises a time-variant system. The system comprises a linear system. The system comprises a nonlinear system. The system comprises a continuous-time system. The system comprises a discrete-time system.

The non-directional input comprises an intensive variable of a conjugate pair of variables. The non-directional input comprises a signal. The non-directional input comprises an externally applied signal. The non-directional input comprises a signal that is intrinsic to the system. The non-directional input comprises an input signal. The non-directional input comprises a noise signal. The non-directional input comprises a control signal. The non-directional input comprises an intermediate signal. The non-directional input comprises a time-independent signal. The non-directional input comprises a time-dependent signal. The non-directional input comprises a continuous-time signal. The non-directional input comprises a discrete-time signal. The non-directional input comprises a deterministic signal. The non-directional input comprises a stochastic signal. The non-directional input comprises more than one signal.

The non-directional input comprises an influence. The non-directional input comprises a chemical influence. The non-directional input comprises an electrical influence. The non-directional input comprises a magnetic influence. The non-directional input comprises a thermal influence. The non-directional input comprises an electromagnetic influence. The non-directional input comprises a flow influence. The non-directional input comprises a pressure influence. The non-directional input comprises a mechanical influence. The non-directional input comprises a gravitational influence.

The non-directional input comprises a combination of two or more influences. The influences are of the same type of influence. The influences are of at least two different types of influences. The influences have the same phase. The influences have different phases with a fixed relationship. The influences have different phases with a varying relationship. The influences have the same frequency. The influences have different frequencies with a fixed relationship. The influences have different frequencies with a varying relationship.

The asymmetry comprises an absence or a violation of a non-isometric symmetry. The asymmetry comprises an absence or a violation of a directional symmetry. The asymmetry comprises an absence or a violation of a reflection symmetry. The asymmetry comprises an absence or a violation of a rotational symmetry. The asymmetry comprises an absence or a violation of a translational symmetry. The asymmetry comprises an absence or a violation of a glide reflection symmetry. The asymmetry comprises an absence or a violation of a rotoreflection symmetry. The asymmetry comprises an absence or a violation of a helical symmetry. The asymmetry comprises an absence or a violation of a scale symmetry. The asymmetry comprises an absence or a violation of two or more symmetries. The asymmetry comprises an externally applied asymmetry. The asymmetry comprises an asymmetry that is intrinsic to the system. The asymmetry comprises a time-independent asymmetry. The asymmetry comprises a time-dependent asymmetry. The asymmetry comprises a one-variable asymmetry. The asymmetry comprises a multi-variable asymmetry. There is more than one asymmetry. All the asymmetries comprise an absence or a violation of the same type of symmetry or antisymmetry. The asymmetries comprise an absence or a violation of two or more types of symmetries or antisymmetries.

The directional effect comprises an extensive variable of the conjugate pair of variables. The directional effect comprises an output signal. The directional effect comprises a noise signal. The directional effect comprises a control signal. The directional effect comprises an intermediate signal. The directional effect comprises a time-independent signal. The directional effect comprises a time-dependent signal. The directional effect comprises a continuous-time signal. The directional effect comprises a discrete-time signal. The directional effect comprises a deterministic signal. The directional effect comprises a stochastic signal. More than one directional effect is achieved. The directional effects are in the same type of variable. The directional effects are in at least two different types of variables. The directional effect comprises doing mechanical work. Doing mechanical work comprises altering the kinetic energy of a system.

The directional effect comprises altering the potential energy of a system. The potential energy comprises gravitational potential energy. The potential energy comprises elastic potential energy. The potential energy comprises chemical potential energy. The potential energy comprises electric potential energy. The electric potential energy comprises electrostatic potential energy. The electric potential energy comprises electrodynamic potential energy. The electric potential energy comprises nuclear potential energy. The potential energy comprises thermal potential energy. The potential energy comprises rest mass energy.

The directional effect comprises doing thermodynamic work. Doing thermodynamic work comprises altering the enthalpy of a system. Doing thermodynamic work comprises altering the entropy of a system. Doing thermodynamic work comprises doing pressure-volume work.

The directional effect comprises doing organizational work. Doing organizational work comprises altering the order of a system. Doing organizational work comprises altering the complexity of a system. Doing organizational work comprises altering the pattern of a system. Doing organizational work comprises altering the structure of a system. Doing organizational work comprises altering the emergent property of a system. Doing organizational work comprises altering the behavior of a system.

The combination comprises one or more actions or steps in a method, one or more elements in an apparatus, one or more parts in a combination of matter, or sub-combinations thereof.

An input modulates a potential energy surface of the system. The potential energy surface is modulated vertically. The potential energy surface is modulated laterally. One or more inputs modulate the potential energy surface of a transition state in the system both vertically and laterally. A transition state of the potential energy surface is modulated. The input directly interacts with the reactants. The input modulates a property of the environment of the reactants. The environment comprises an active pocket of an enzyme. The environment comprises a supramolecular structure. The supramolecular structure comprises an aptamer. The supramolecular structure comprises a zeolite. The supramolecular structure comprises a polymer. The supramolecular structure comprises a carbon nanotube. The system comprises an influence mediator. The influence mediator comprises a charged bead or a magnetic bead. The influence mediator comprises a linker.

The system is designed using ab initio simulations, catalytic antibodies and/or in vitro evolution. The system comprises a modification to enhance an input. The modification comprises attaching an enzyme or a supramolecule to a surface of a reaction chamber with an electrical double layer formed at that surface. The modification comprises coating a surface of a reaction chamber with a flexible substrate.

In general, in an aspect, the invention features, an apparatus comprising a site for a reaction, and a device interacting with a system that has a perturbation. In the perturbed system a non-directional input is applied to a first variable of the system. Based on an asymmetry of the perturbed system, a directional effect is achieved in a second variable of the system, the first and second variables comprising a conjugate pair of variables. At least one of the following pertains: the interaction occurs other than by an apparatus and other than in a way that actually achieves the directional effect, or the conjugate pair is other than position and momentum, or the input or the asymmetry is in a dimension other than spatial coordinates, or the directional effect is other than one-dimensional translational motion and other than one-dimensional rotary motion.

The reaction comprises a chemical reaction. The reaction comprises a biochemical reaction. The device comprises one or more controlled inputs. An input comprises a controlled voltage. An input comprises a controlled mechanical force. An input comprises a controlled temperature. An input comprises a controlled pressure. The device comprises a surface.

The directional effect comprises converting a type of non-chemical energy into another type of non-chemical energy. Electrical energy is converted into rotary power or mechanical work. Rotary power or mechanical work is converted into electrical energy. The directional effect comprises converting a type of chemical energy into a type of non-chemical energy. A chemical fuel energy is converted into a non-chemical energy. A chemical energy is converted into electrical energy. A chemical energy is converted into rotary power or mechanical work.

The directional effect comprises converting a type of non-chemical energy into a chemical energy. Electrical energy is converted into a chemical energy. A non-chemical energy is converted into a high energy density chemical fuel. The high energy density chemical fuel comprises methane. The high energy density chemical fuel comprises ethane. The high energy density chemical fuel comprises hydrogen. A non-chemical energy is converted into a biofuel. The biofuel comprises methanol. The biofuel comprises ethanol. A non-chemical energy drives a chemical fuel process. The chemical fuel process comprises gasoline cracking The chemical fuel process comprises gasoline synthesis.

The directional effect comprises converting a type of chemical energy into another type of chemical energy. The conversion reaction comprises CO₂ reduction. The conversion reaction comprises glucose to fructose conversion. The conversion reaction comprises ethylene production.

The directional effect comprises manipulating a chemical reaction. The chemical reaction is an intermediate chemical reaction. The chemical reaction is a biochemical reaction. The manipulation comprises controlling a direction of the reaction. The manipulation comprises altering a final substrate and/or a final product concentration or the ratio of the two concentrations. The manipulation comprises doing work on the system that the system would otherwise not do, including against or along other influences and/or gradients. The manipulation comprises catalyzing the reaction. The manipulation comprises specific enhancement and/or suppression of reactions and/or chemical paths. The manipulation comprises increasing, decreasing, or reversing a spontaneity of the reaction. The manipulation comprises changing a probability of a specific path and/or product, relative to another alternative path or product, to change a yield of the specific path and/or product.

A result of the method comprises new mixtures and/or products. A new mixture of product produced by applying the method.

The system is used in chemical manufacturing. The system is used in industrial processing. The system is used in catalysis. The system is used in chemical fuel production. The system is used in electricity generation. The system is used in rotary power or mechanical work generation. The system is used in energy storage. The system is used in reduction of undesired chemicals. The undesired chemical comprise greenhouse gases.

The directional effect comprises altering the negentropy of a system.

The system comprises a self-organizing system. The self-organizing system comprises a protein. The self-organizing system comprises a self-assembling molecule. The system comprises a process. The process comprises cell signaling. The process comprises homeostasis. The process comprises a developmental stage of a living organism.

The system is used in basic life science research. The system is used in medicine. The system is used in a synthetic life process or a product. The directional effect comprises transporting an object. The transportation is against an opposing force and/or gradient. The object comprises a micro-object. The object comprises an ion. The object comprises a molecule. The object comprises a biomolecule. The object comprises a biological cell. The object comprises a macro-object.

The object comprises a transportation vehicle. The system is used in mechanics. The system is used in biological transportation. The system is used in chemical transportation. The system is used in vehicular transportation.

The directional effect comprises altering a property of an object and/or a process. The property comprises structure. The property comprises complexity. The property comprises strength. The property comprises elasticity. The property comprises weight.

The system is used in material science. The system is used in manufacturing.

The directional effect comprises altering the electromagnetic property of an object and/or a process. The system is used in electronics. The system is used in communications.

The system comprises a pump that is driven by the dynamics of the system. The pump alters the concentration of an object. The pump alters the transfer speed of an object.

In general, in an aspect, a combination comprises interaction with a system that has a perturbation. In such perturbed system, an input is applied to a first variable of the system. Based on an asymmetry of the perturbed system, a directional effect is achieved in a second variable of the system. The first and second variables comprise a conjugate pair of variables.

In general, in an aspect, a combination comprises interaction with a system that has a perturbation. In such perturbed system, an input is applied to a first variable of the system. Based on an asymmetry of the perturbed system, a directional effect is achieved in a second variable of the system. The first and second variables comprise a conjugate pair of variables other than position and momentum.

In general, in an aspect, a combination comprises non-physical interaction with a system that has a perturbation. In such perturbed system, an input is applied to a first variable of the system Based on an asymmetry of the perturbed system, a directional effect is achieved indirectly in a second variable of the system, The first and second variables comprise a conjugate pair of variables.

In general, in an aspect, a combination comprises interaction with a system that has a perturbation. In such perturbed system, an input is applied to a first variable of the system. Based on an asymmetry of the perturbed system, a directional effect is achieved in a second variable of the system. The first and second variables comprise a conjugate pair of variables. The input or the asymmetry is in a dimension other than spatial coordinates.

In general, other aspects include combinations of these features useful for producing particular products, such as hydrogen.

These and other aspects and features, and combinations of them, can be expressed as systems, methods, compositions of matter, manufactures, methods of doing business, means or steps for performing functions, program products, methods of manufacture, methods of use, combinations, and in other ways.

Other aspects and features will be apparent from the following description and from the claims.

### DESCRIPTION

FIGS. 1A, 1B, and 1C illustrate a Brownian ratchet.

FIGS. 2A and 2B illustrate a flashing ratchet.

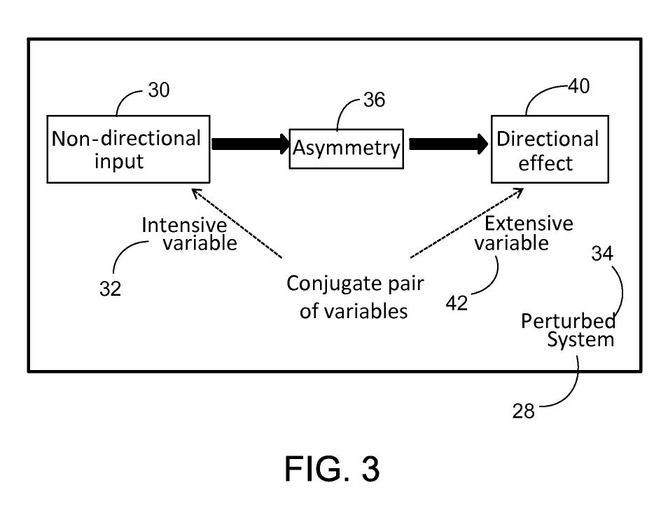

FIG. 3 is a block diagram of a system.

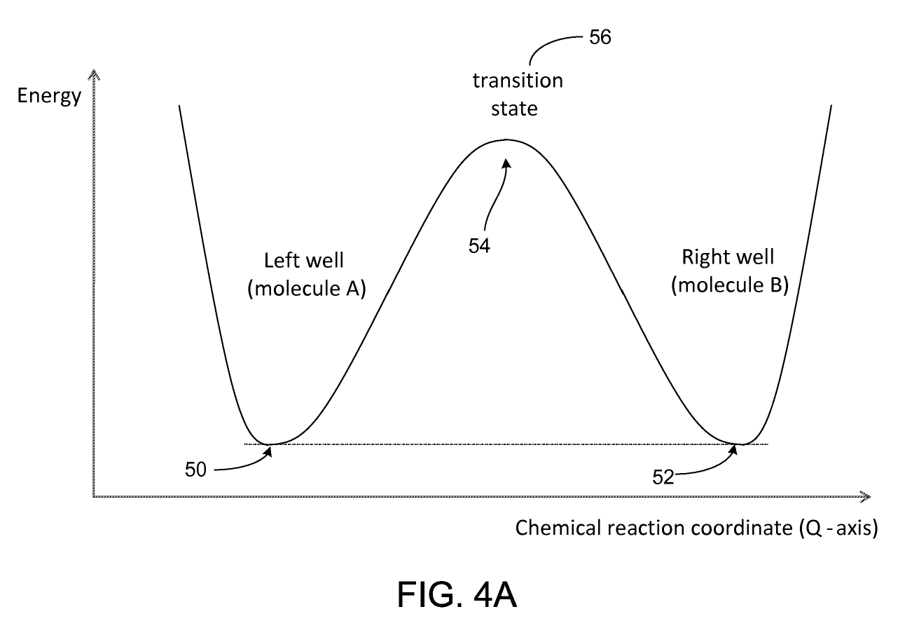

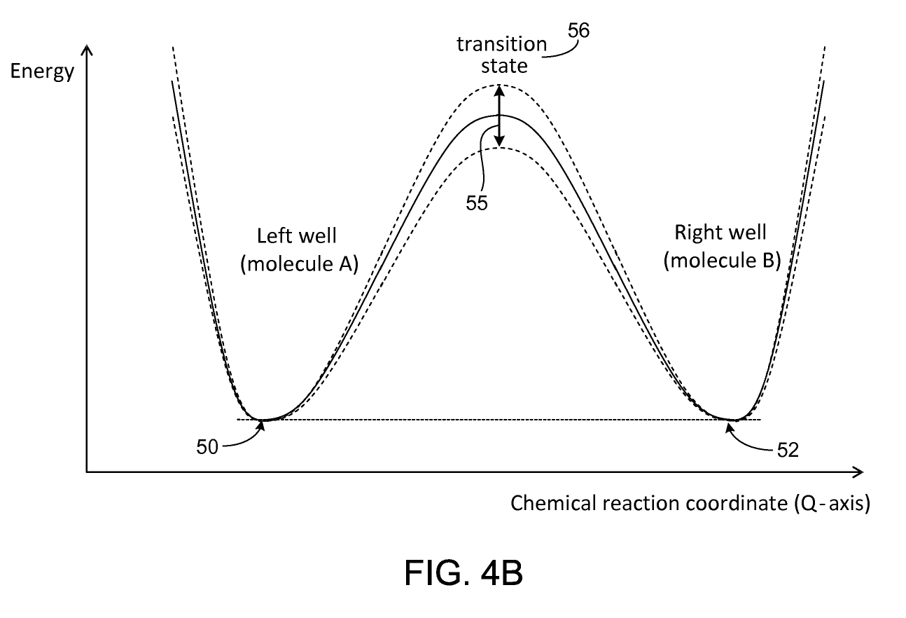

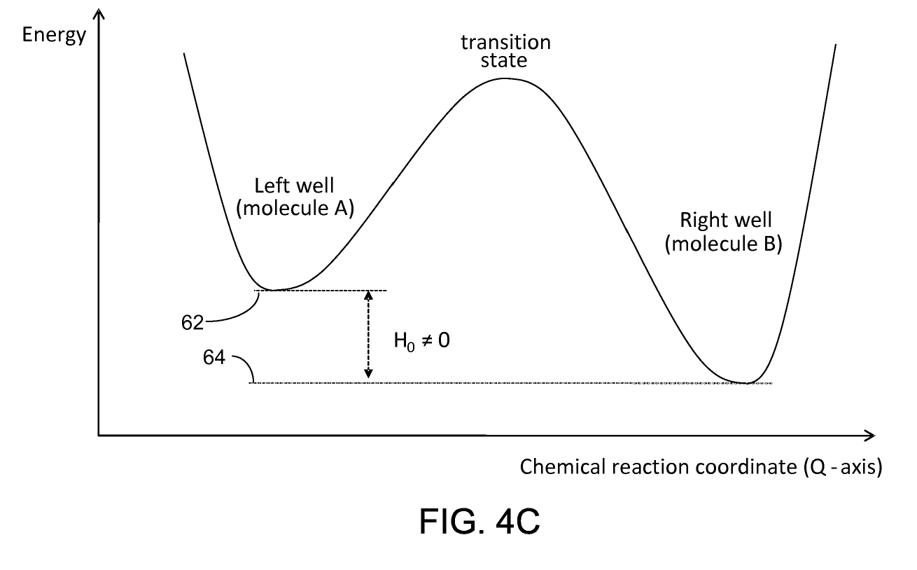

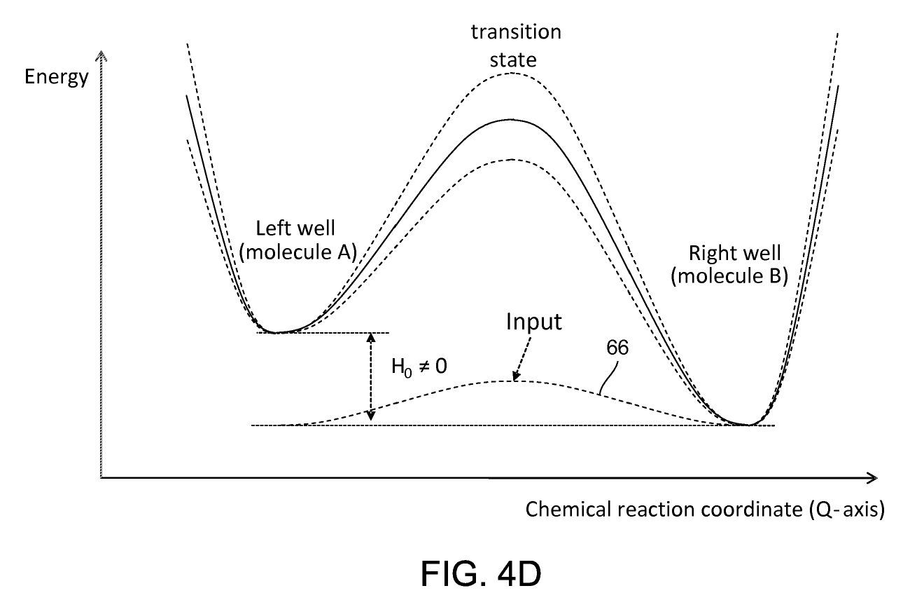

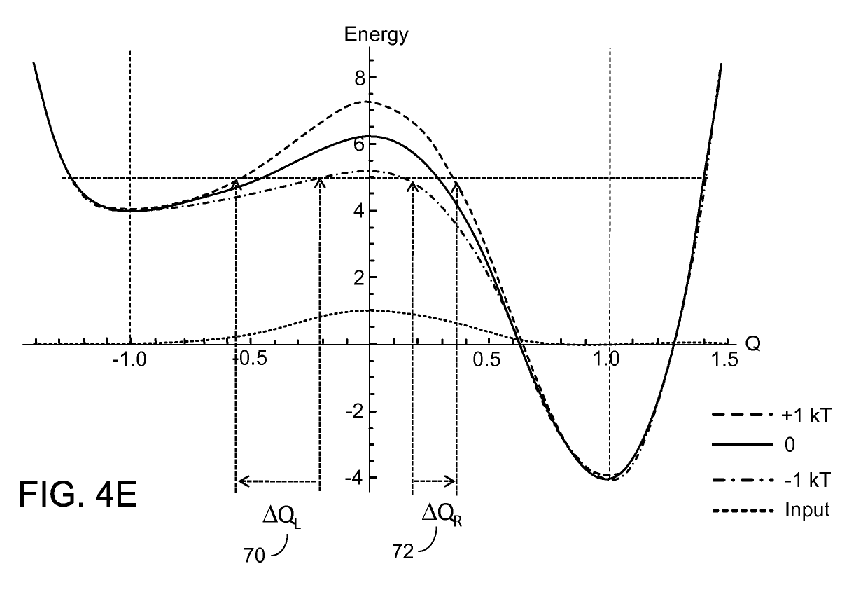

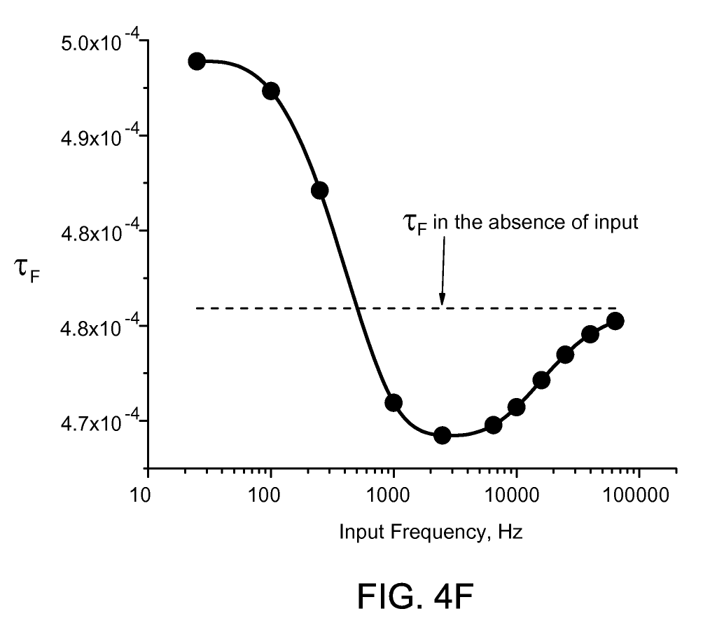

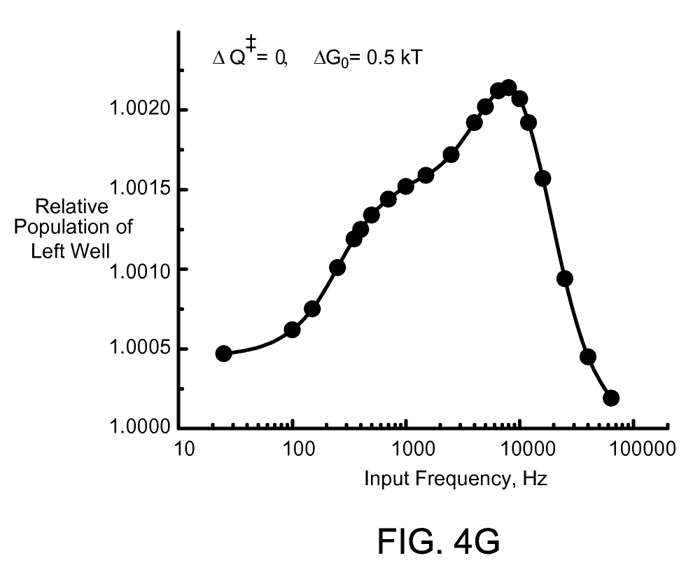

FIGS. 4A, 4B, 4C, 4D, 4E, 5A, 5B, 5C, 6A, 7A, 7C, 9A, 9B, and 9D are graphs of energy versus chemical reaction coordinate (Q-space).

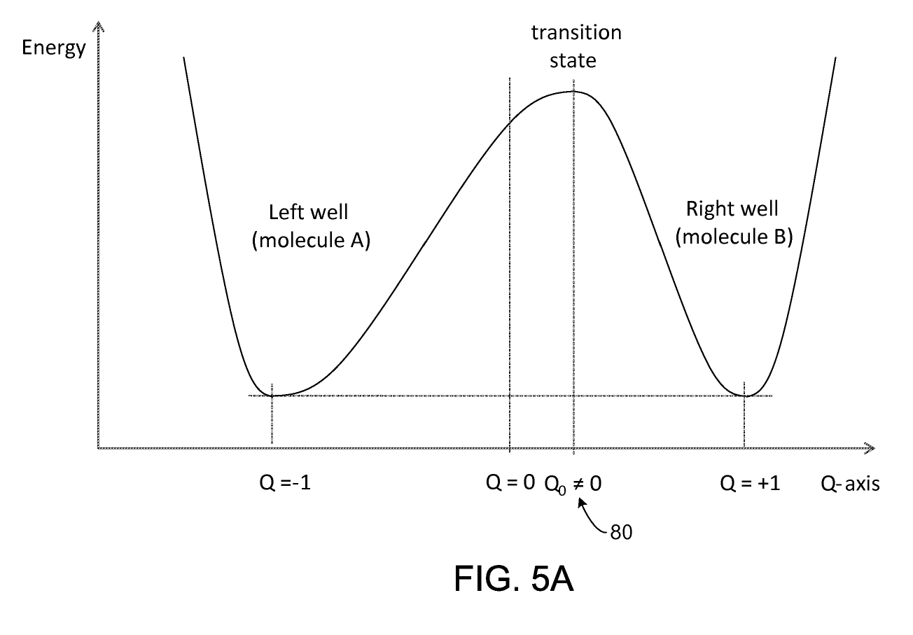

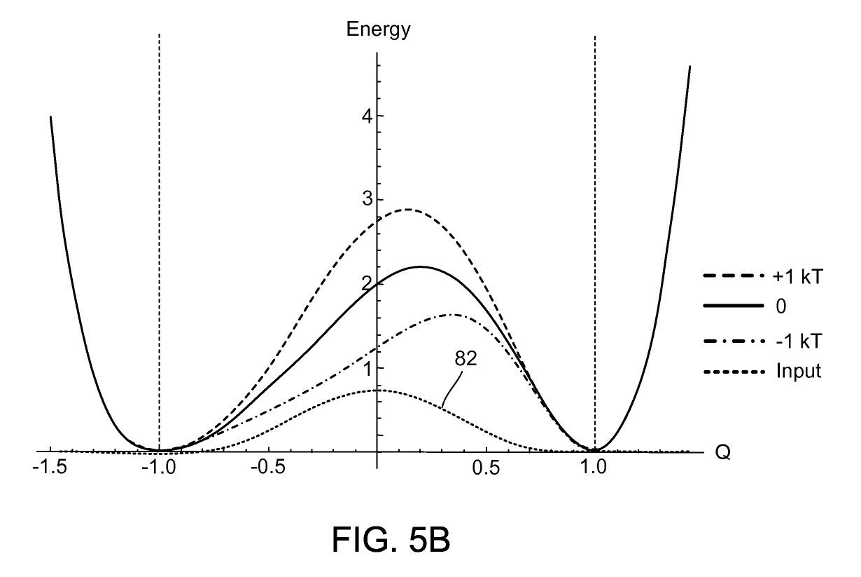

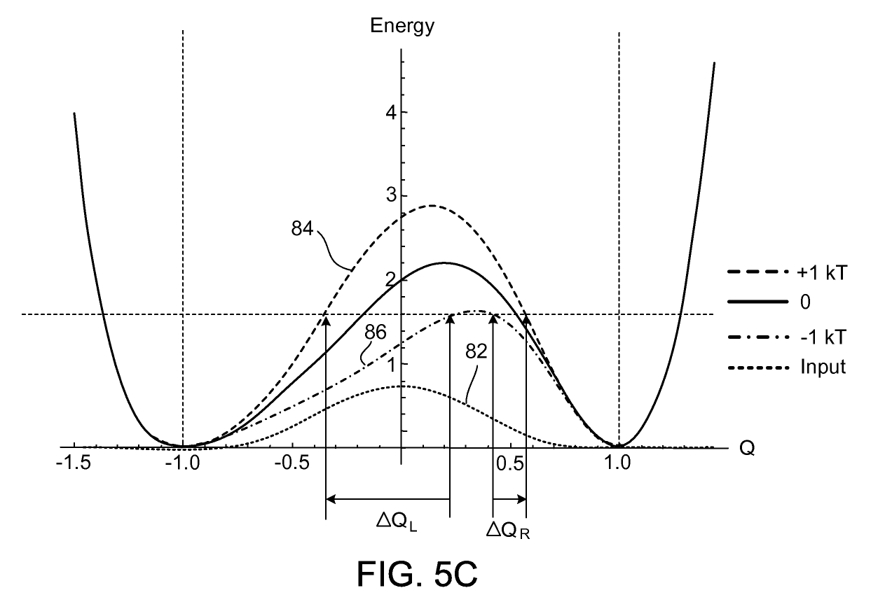

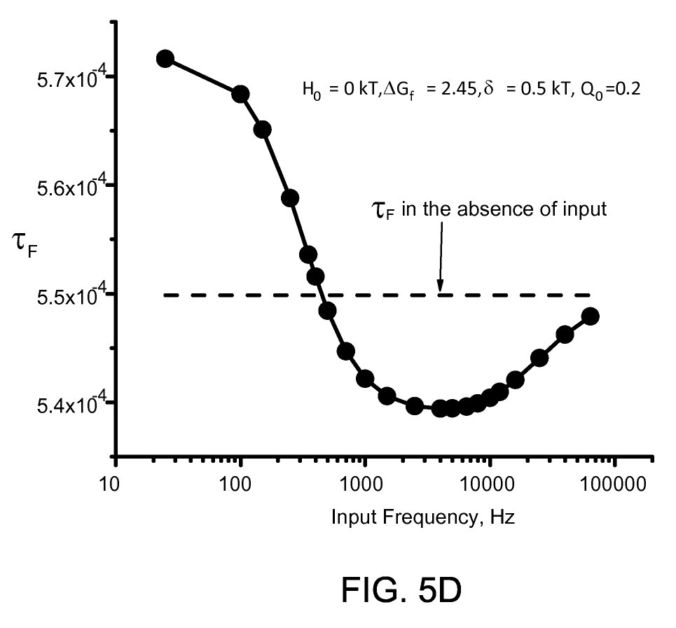

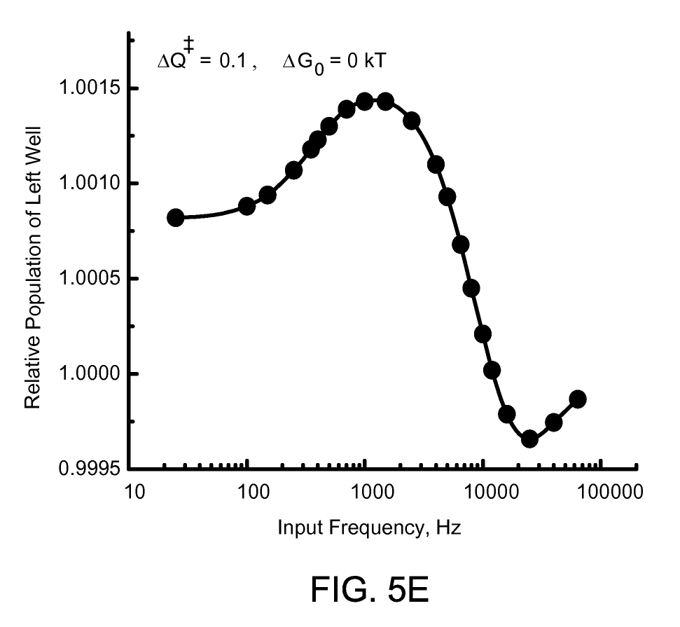

FIGS. 4F and 5D are plots of time constants versus frequency.

FIGS. 4G, 5E, 9C, and 9E are plots of relative populations of molecules versus frequency.

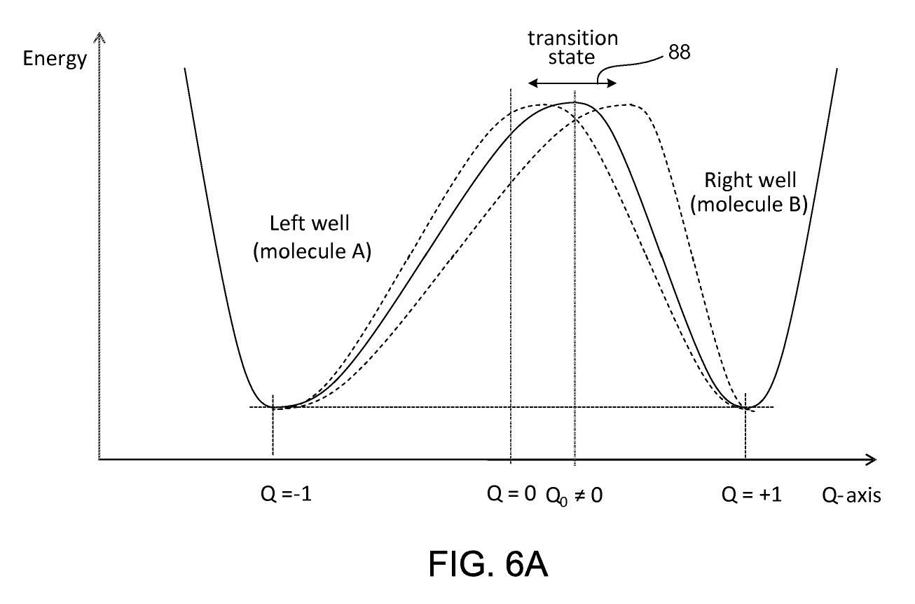

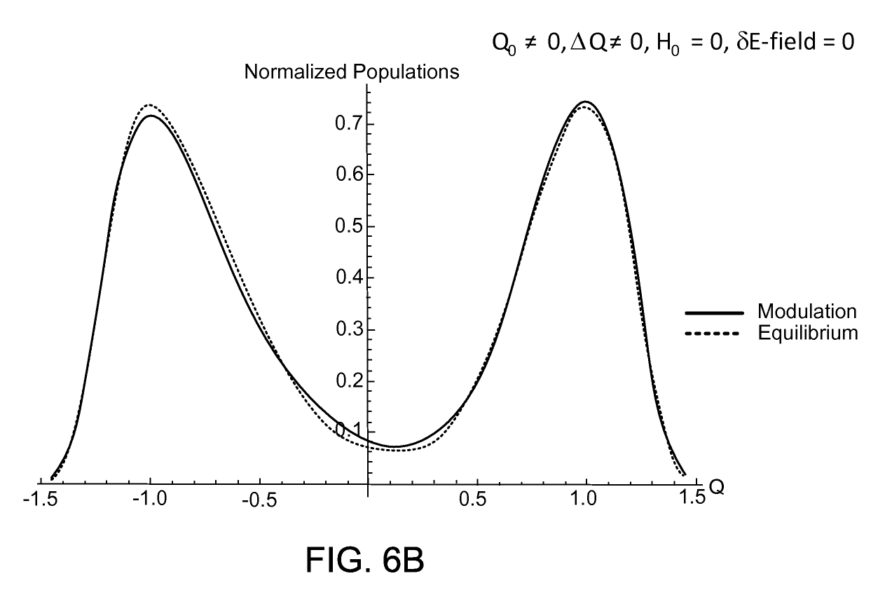

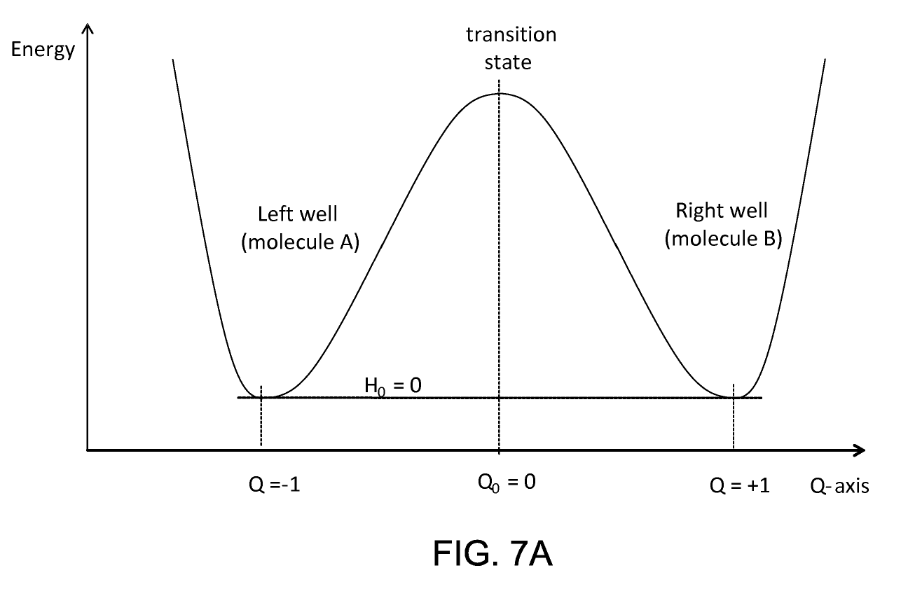

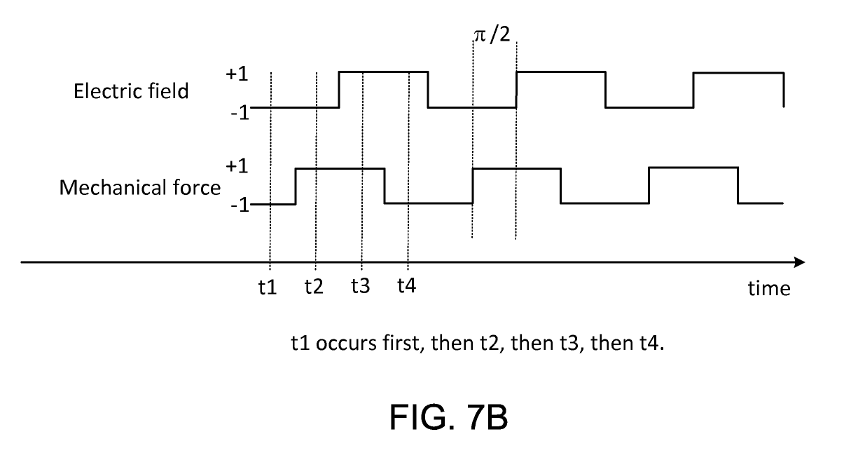

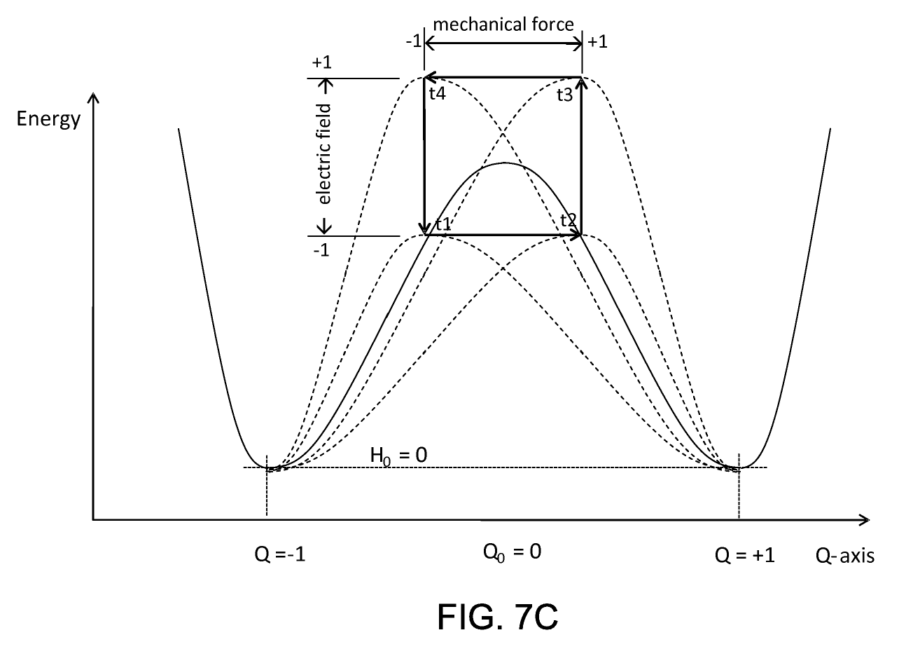

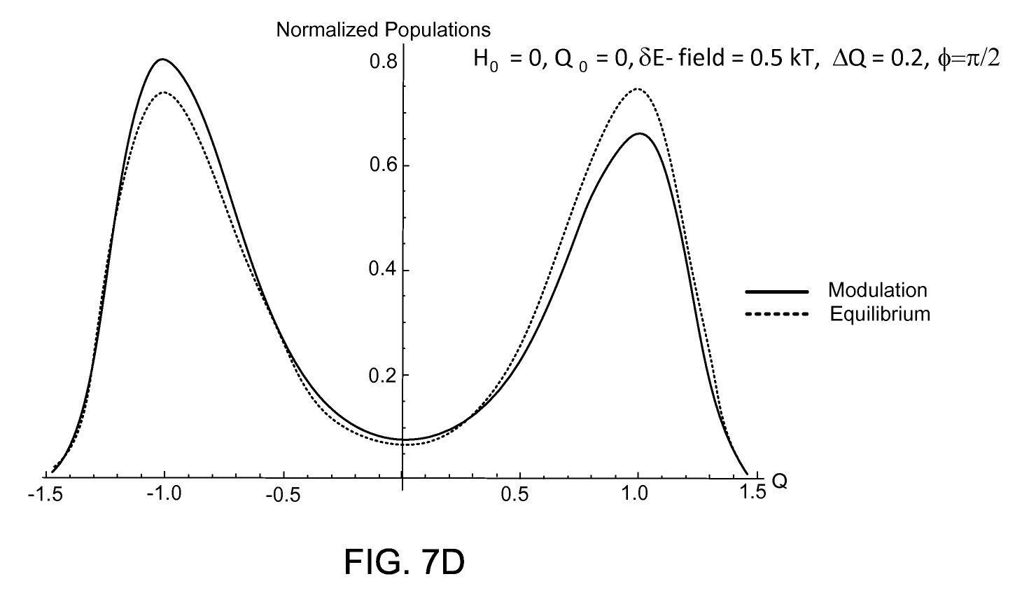

FIGS. 6B and 7D are plots of normalized population distributions of molecules versus chemical reaction coordinate (Q-space).

FIG. 7B is diagram of a signal waveform versus time.

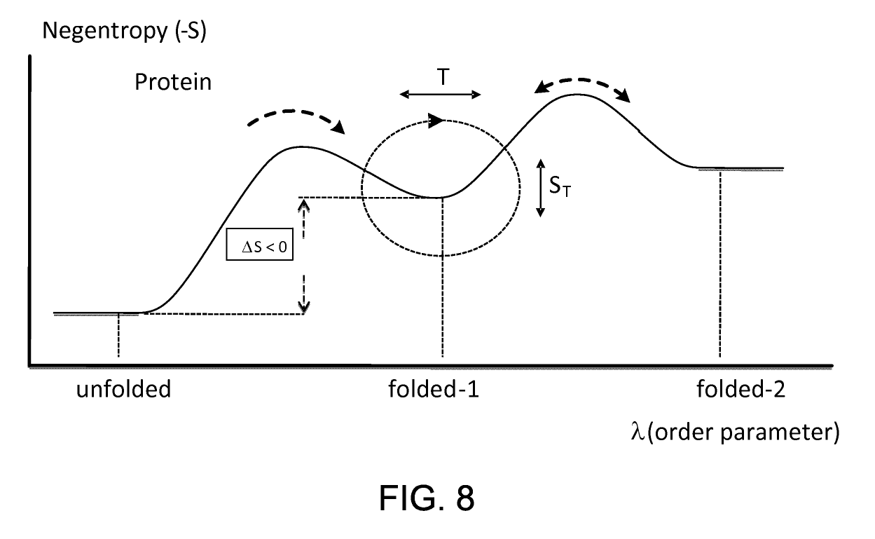

FIG. 8 is a diagram of negentropy versus order parameter.

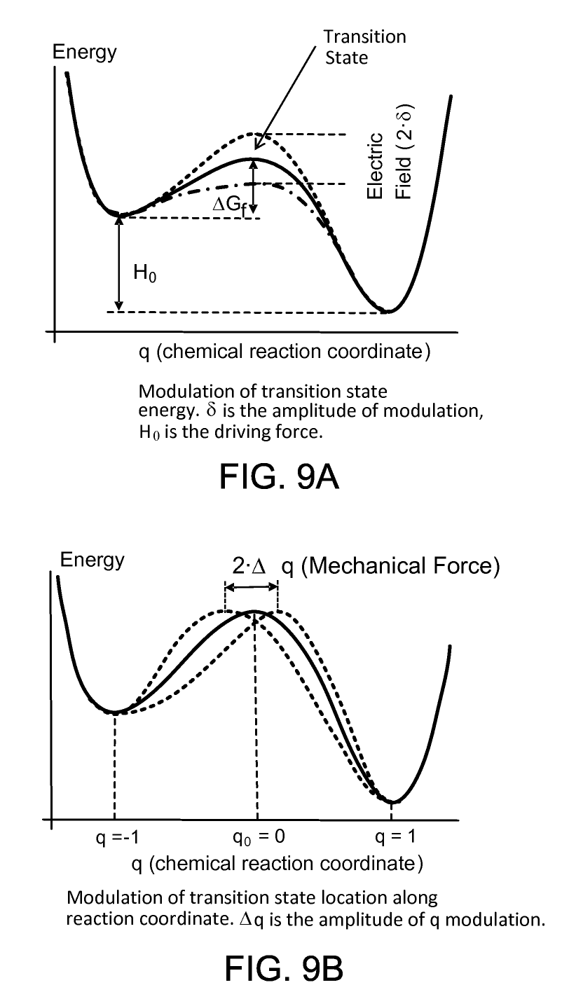

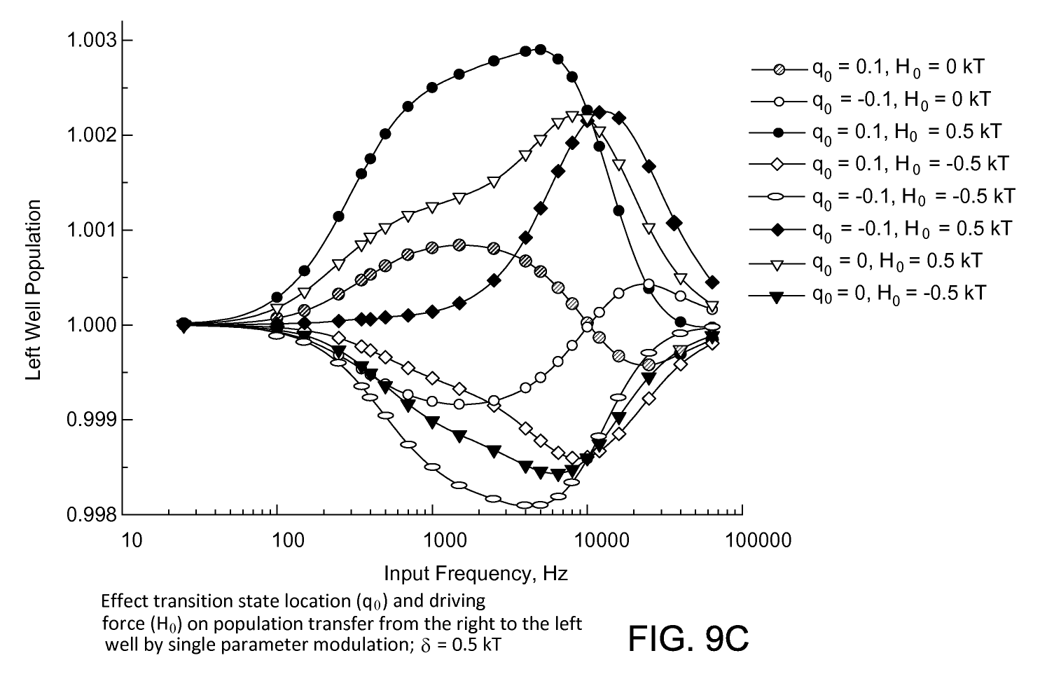

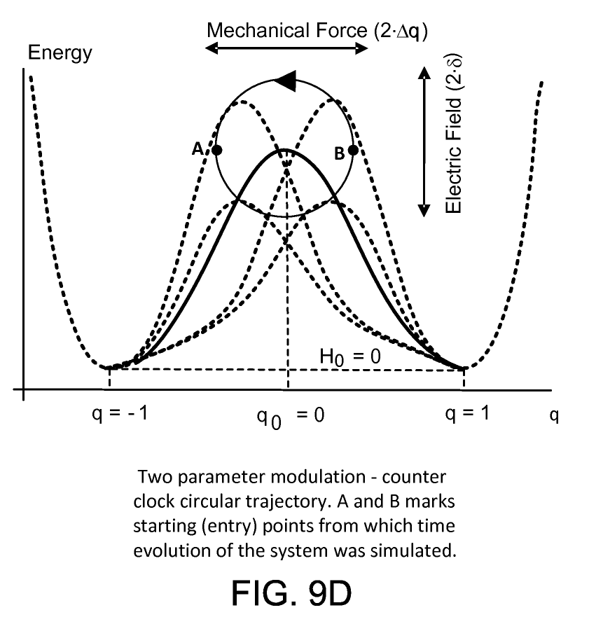

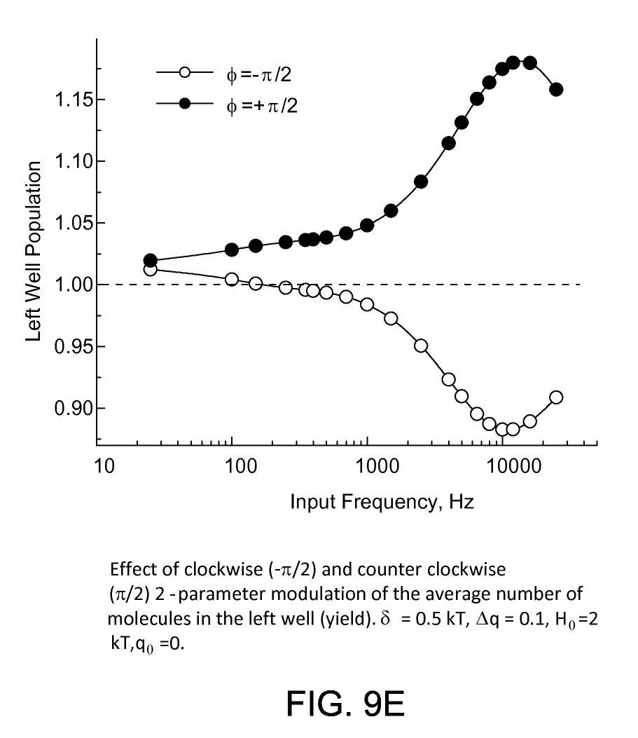

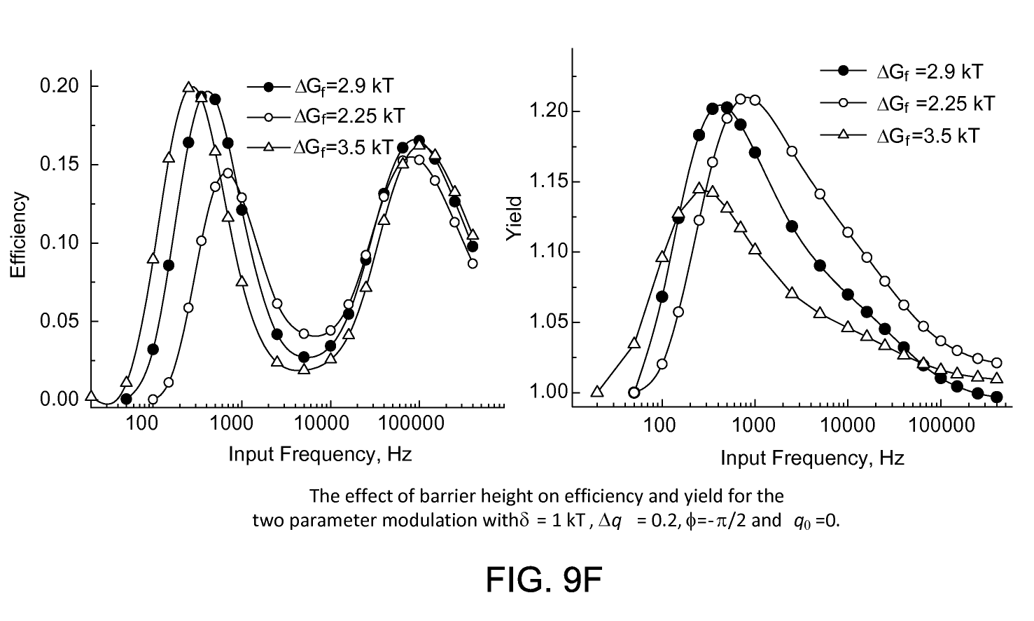

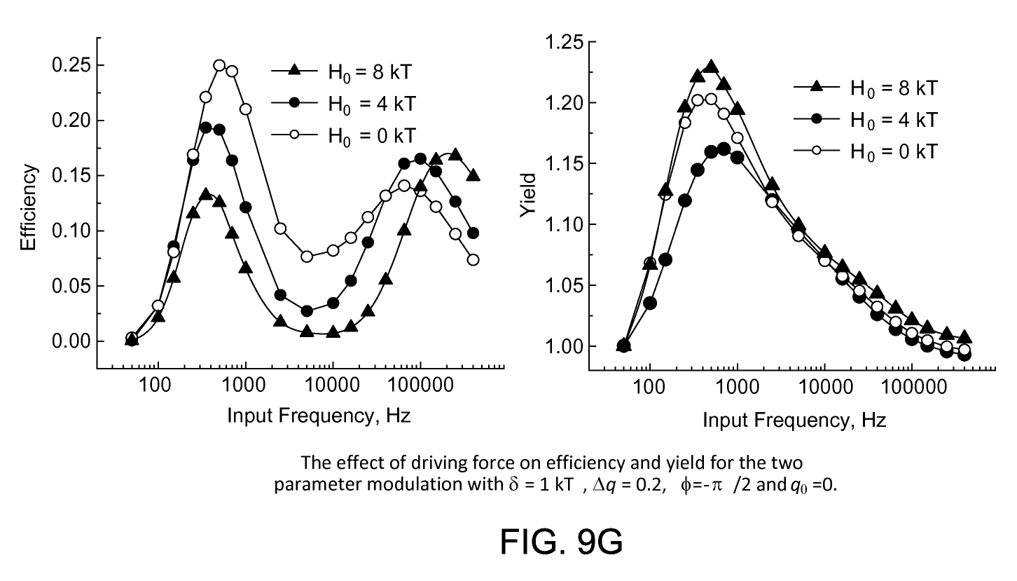

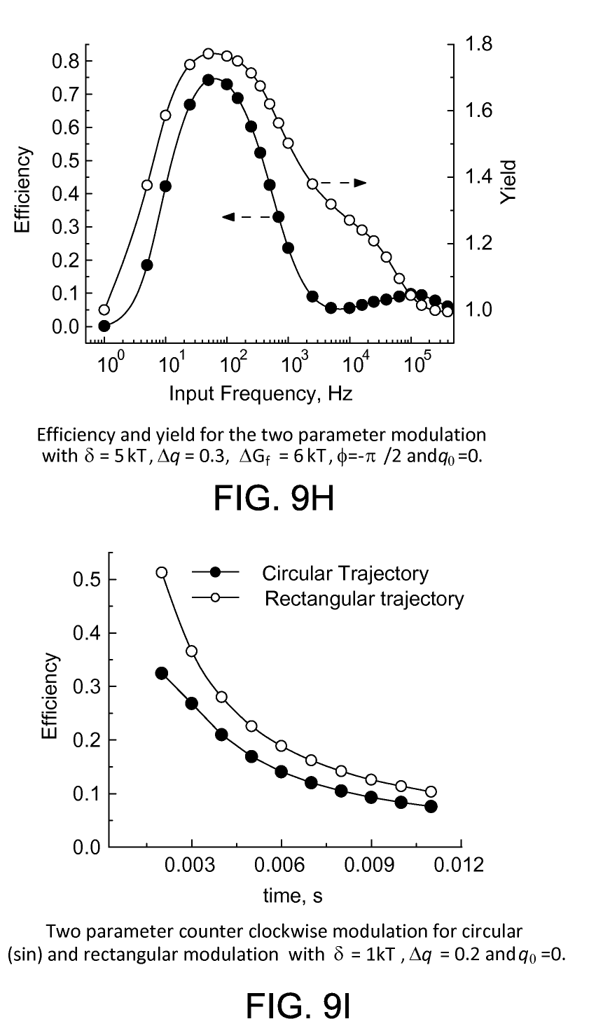

FIGS. 9F, 9G, 9H, and 91 are diagrams of efficiency and yield versus frequency.

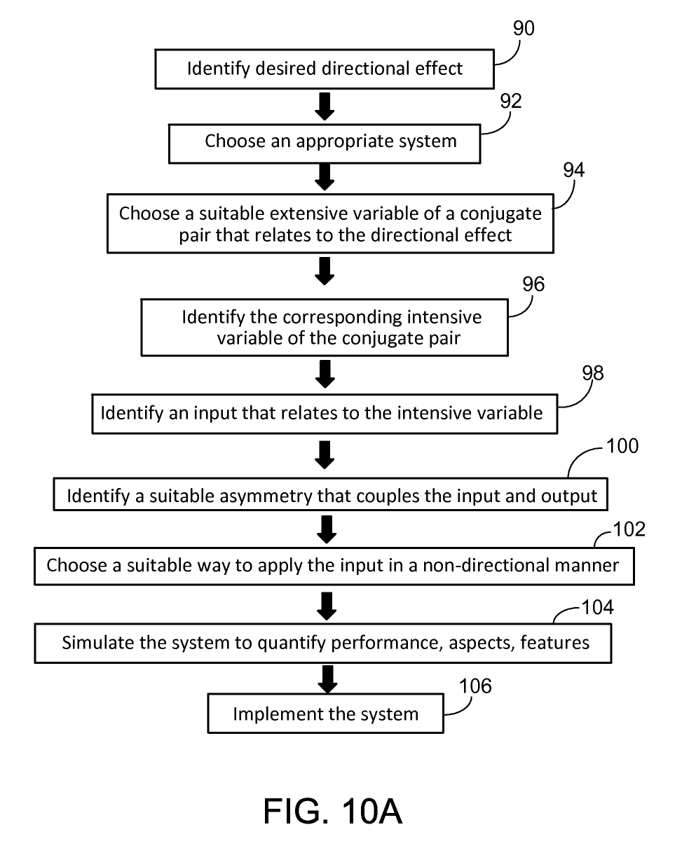

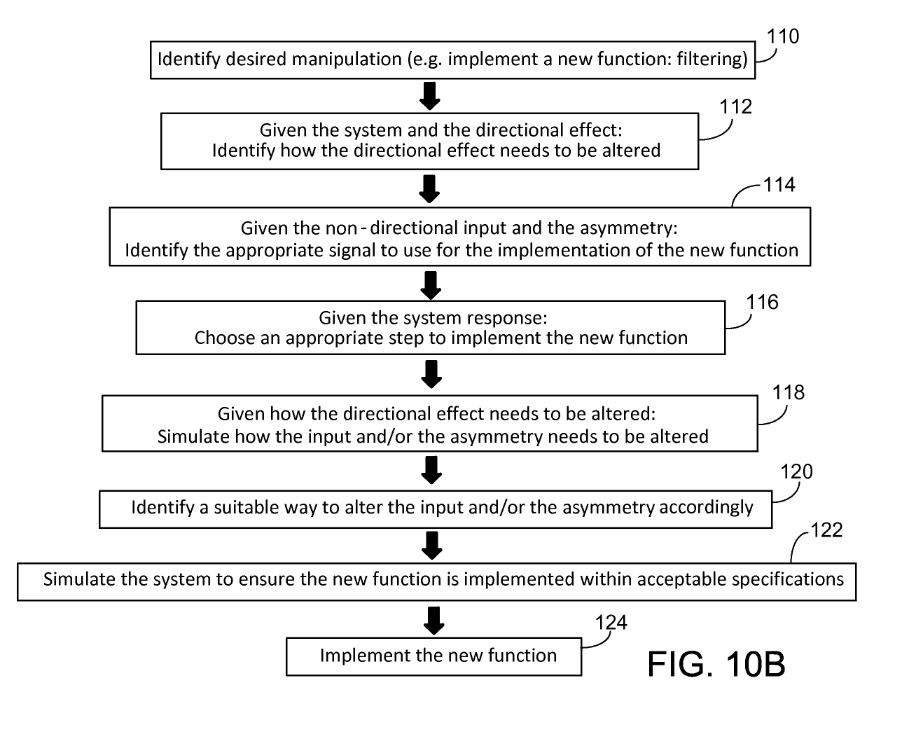

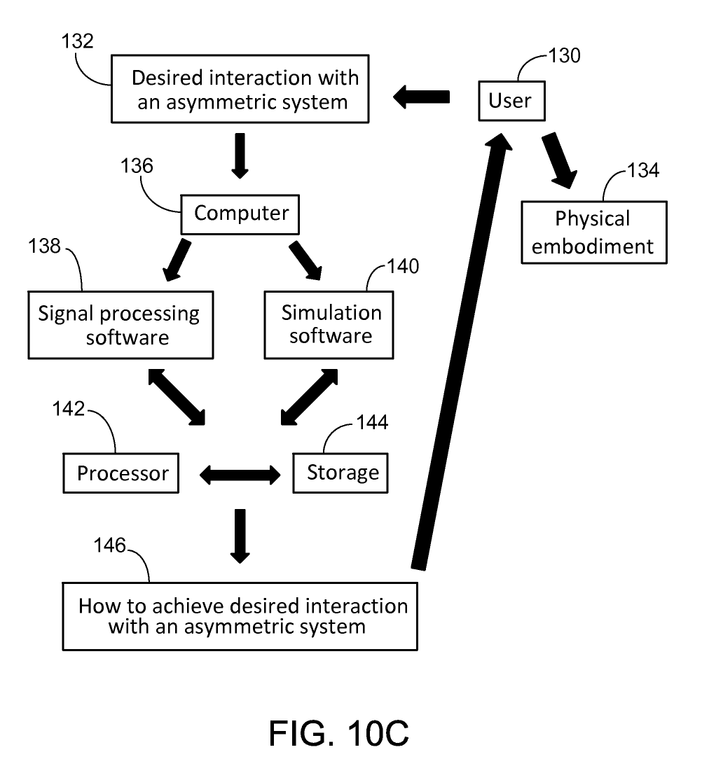

FIGS. 10A, 10B, and 10 C are flow diagrams of an interaction with a system.

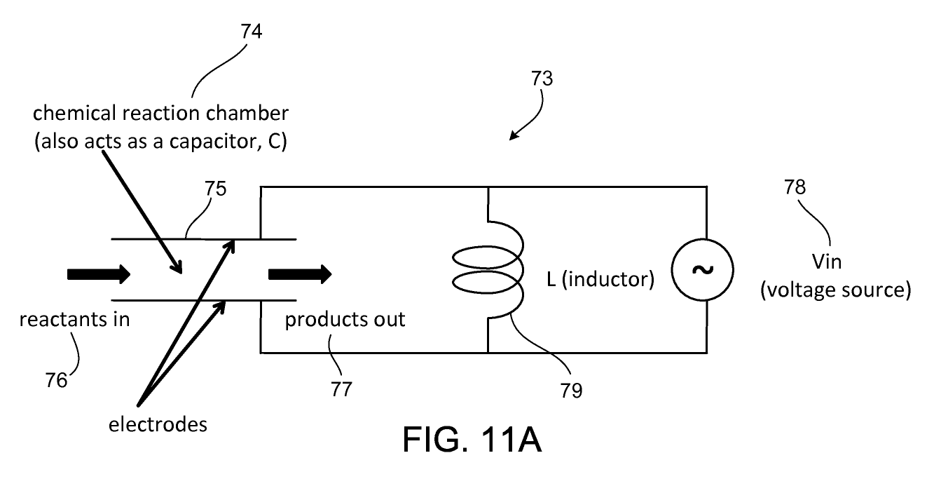

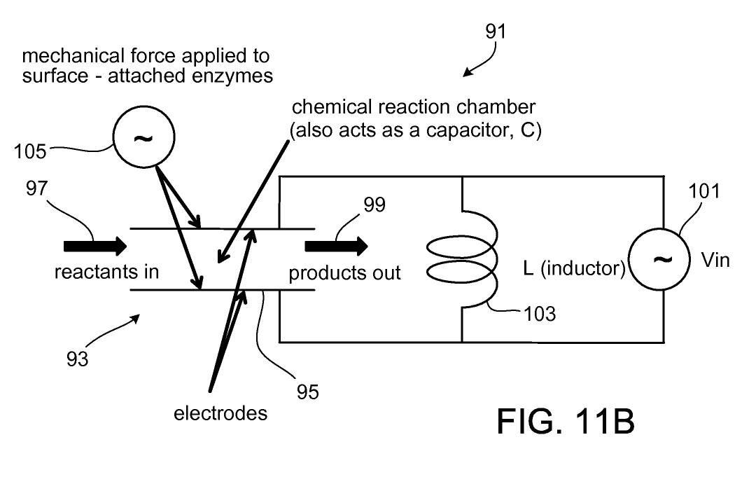

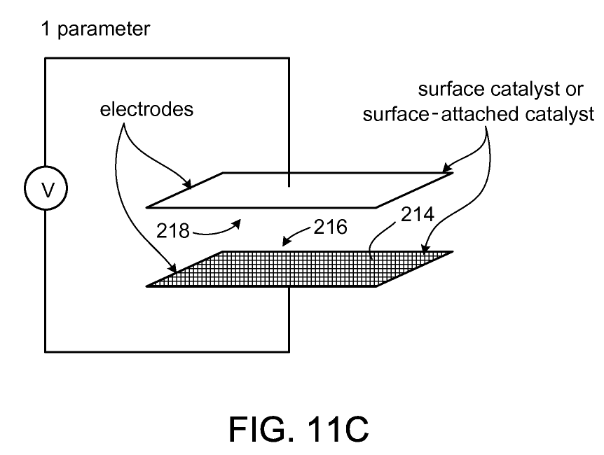

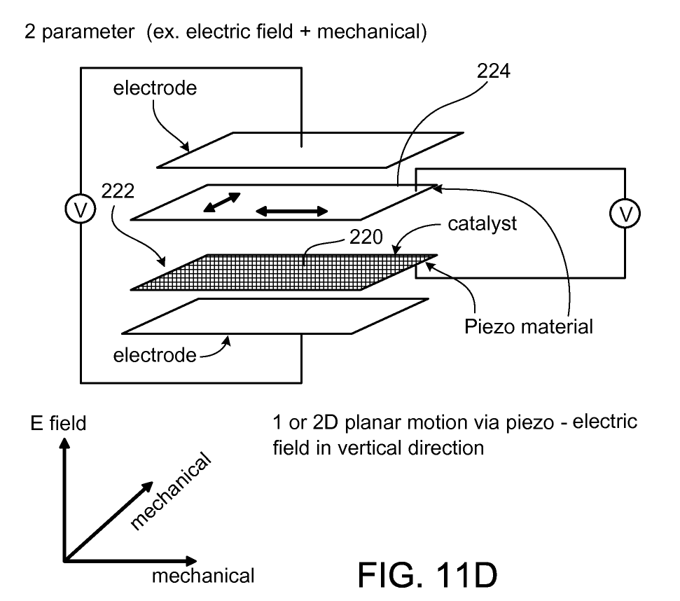

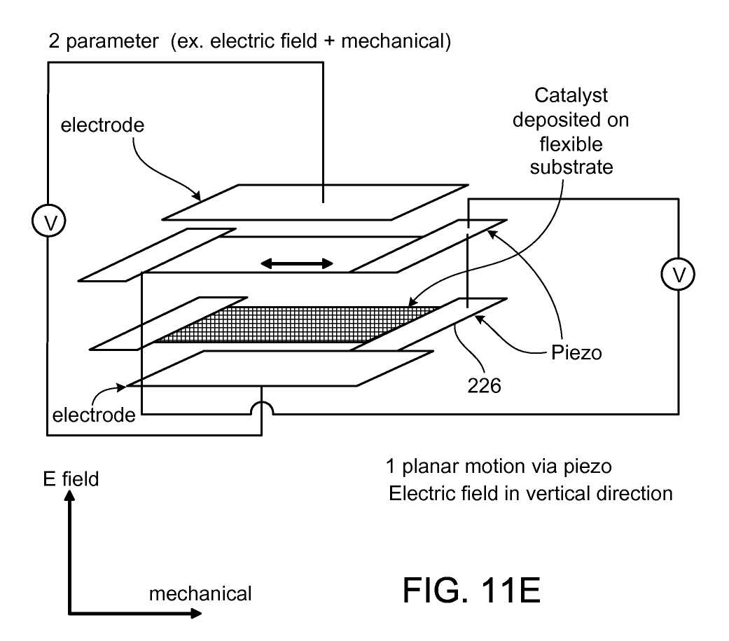

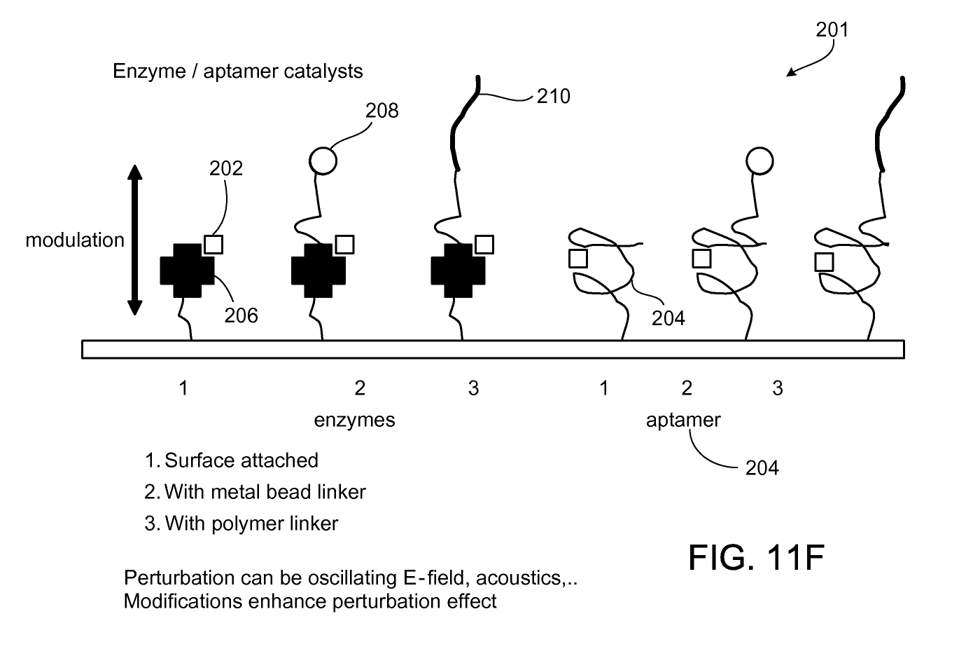

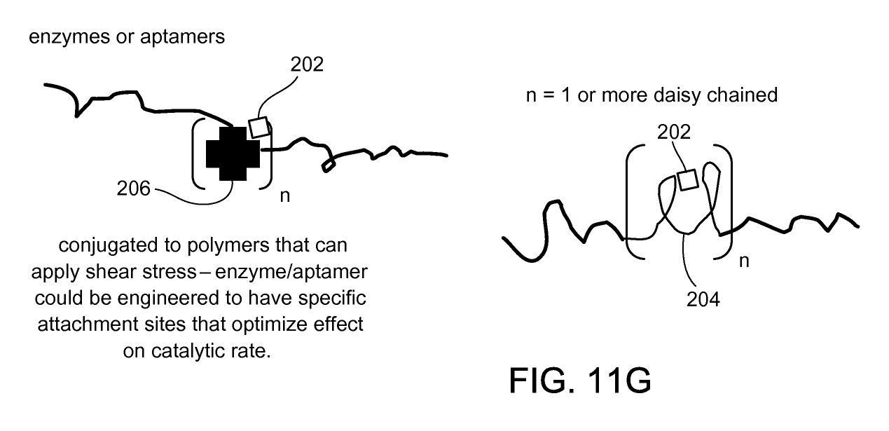

FIGS. 11A, 11B, 11C, 11D, 11E, and 11F are drawings of apparatus.

FIG. 11G is a schematic diagram of enzymes/aptamers with polymers.

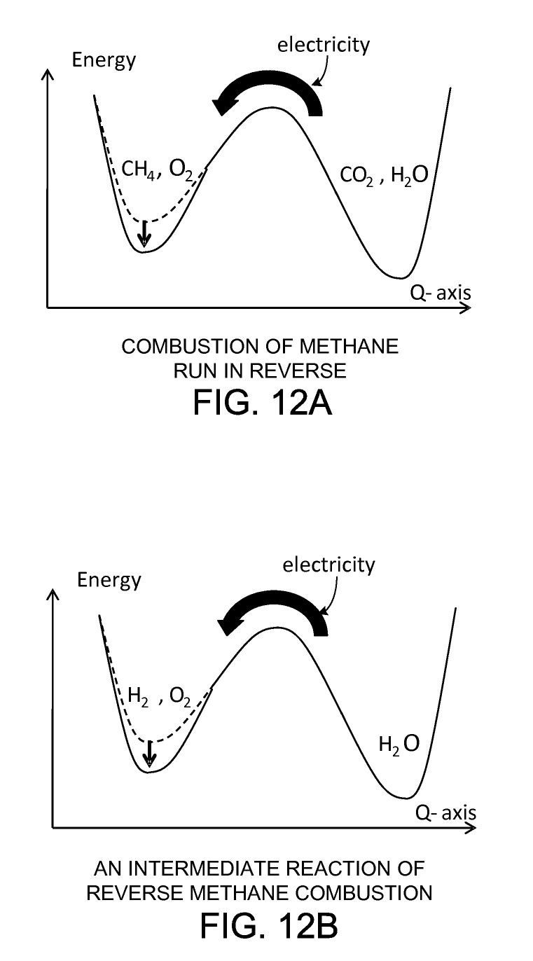

FIGS. 12A and 12 B are graphs of chemical reactions, driven by electrical energy being transduced into chemical energy.

Here, we describe a broad concept and a broad understanding of a new energy transduction technique, in which a non-directional input or influence (we often use the words interchangeably) achieves a directional effect in a system.

We use the term non-directional very broadly to include, for example, that an average of an input or influence applied to the system by a signal, over one or more ranges of interest for a dimension or dimensions along which one or more effects is to be achieved, is zero.

And we use the term directional very broadly to include, for example, that an average influence applied to the system by a signal, over one or more ranges of interest for the dimension or dimensions along which the one or more effects is to be achieved, is not zero.

We use the term influence very broadly to include any kind or nature of influence, including, for example, a force, a torque, or an event that alters a state of a system or a property of a system, or any combination of influences.

We use the term signal also very broadly to include any function of one or more independent variables. The signal may contain, express, or imply information about a behavior or nature of a phenomenon. In some examples, the signals can be mathematical or abstract or other representations or implementations.

We use the term system very broadly to include, for example, two or more interacting or interdependent entities, real or abstract, which in some examples may form an integrated whole. The term system may also include, for example, any process that results in the transformation of signals. Thus, in some examples, a system has an input signal and an output signal which is related to the input through a system transformation. The term system may further include, for example, one or more of a subset of a system (e.g. a subsystem), an object or an element in a system, or a relationship between objects or elements of a system or its surroundings.

We use the term transduction very broadly to include, for example, the conversion, translation or alteration of one form of energy into another form of energy.

The energy which is subject to the transduction and the energy into which it is transduced can take a very wide variety of possible forms and amounts, in some cases different than, more effective or efficient than, or in other ways better than would be the case for known energy transduction techniques. We also use the term energy very broadly to include, for example, internal energy, negentropy (i.e. negative entropy), or a property of a system that is conserved and that can be related to an energy term (e.g. momentum, volume, enthalpy, entropy).

As an example, this new energy transduction technique can be used to enable a conversion from energy in one form (e.g., chemical) into energy in another form (e.g., electrical or another chemical form) without requiring an intermediate step of energy conversion to heat energy. This energy conversion is therefore more efficient, can be simpler and less expensive to implement, and can apply to an extremely broad range of energy conversion regimes. In some examples of the new energy transduction, the order and/or the complexity of a system may be increased (e.g. the entropy of the system may be reduced), which may lead to, for example, self-organization or an emergent property or behavior.

This new energy transduction technique offers the potential to change and benefit broad areas of activity and many disciplines.

Here, among other things, we describe interactions with a system. We use the term interaction very broadly to include, for example, using a system to achieve a desired outcome, designing a system for the purpose of achieving a desired outcome, manipulating a system (which may include analyzing the system, optimizing the system, or implementing a new function in the system), an apparatus that itself embodies all or part of the system, or any combination of those.

We use the term apparatus very broadly to include, for example, any tangible structure, instrument, appliance, device, machine, mechanism, setup, computer, software, network, equipment, or other thing of any kind.

The interactions with systems that we contemplate in this description include designing systems, manipulating systems, interacting based on signal processing, actually achieve a directional effect, and applications of the interactions. Any of these and combinations of these can be achieved with apparatus, including but not limited to apparatus examples that we describe here.

In many cases, the apparatus can be implemented in a wide variety of kinds of computing hardware, software, firmware, or combinations of them, in many cases with the aid of a wide variety of communication networks, user interfaces, interface devices, operating systems, databases, processes, process control and monitoring systems, and user applications.

An interaction in a system 28 may have multiple steps (FIG. 3). In some examples, first, a non-directional input 30 is applied to a variable 32 of the system. As such, in the absence of a load (on which the input force is to act to achieve a desired effect, e.g. do work), the signal associated with the input averages to zero, and thus the force applied to the system is non-directional.

Also, the system is a perturbed system 34. We use the term perturbed very broadly to include any perturbations of the system, including perturbations that are caused by the input 30 to the system, or noise that is intrinsic to the system, or another kind of signal in the broadest sense.

Then, there's an asymmetry 36 in the system. In contrast to asymmetry, within the term symmetry, we very broadly include, for example, any invariance of values (e.g., a lack of any perceptible change) under a transformation over a range of interest. Also, within the term antisymmetry, we very broadly include, for example, a symmetry in which the values under the transformation are of opposite sign or sense. And, by asymmetry, we mean very broadly, for example, an absence or a violation of a symmetry or of an antisymmetry or of both.

When the non-directional input is applied to the perturbed system, based on an asymmetry of the system, the result (which we sometimes refer to as the system's output) is a directional effect 40 that occurs at least in part in a second variable of the system. Therefore, the system outputs an effect, for example, a desired effect (e.g., does work) in a directional manner.

We call each such system an asys (ASYmmetric System or Asymmetric SYStem), and we sometimes refer to a given asys with reference to its non-directional input, its asymmetry, and its directional effect, in that order. For example, if an electric field (E) is applied as an input, there's an asymmetry along a Q-axis of a chemical reaction (Q), and a particle number (N) of a particular output molecule (i.e., chemical yield) is changed, we sometimes call it an E-Q-N-asys.

For example, we described a Q-ratchet in the provisional patent application Ser. 61/090,028, filed Aug. 19, 2008, cited above. A Q-ratchet is an example of the systems illustrated in FIG. 3.

Here, we elaborate on a Q-ratchet system, and provide examples for some of its variations.

In some examples, let's assume we have a chemical system (which we define very broadly and includes, for example, but is not limited to, any chemical reaction having one type of molecule in a left energy well 50 (molecule type A), another type of molecule in a right energy well 52 (molecule type B), and an energy barrier 54 between the two wells) (FIG. 4A).

Also assume that we apply a sinusoidal electric field 55 that modulates a potential energy of a transition state 56 of the molecules in the chemical reaction (FIG. 4B). Although the field has an effect on the transition state, the effect of the modulated electric field on the potential energies of the energy minima of the left well and of the right well approaches zero. At any point along the Q-axis, if we take the average of the force applied by the electric field to the system over time, we get zero, which means the electric field input is non-directional in time. Note that in this application, we often refer to any input that modulates the transition state vertically (e.g., the energy level of the transition state) as an electric field, even though strictly speaking, the input that modulates the energy level of the transition state need not be electrical; it could be a wide variety of other inputs. For example, given a particular system, it may not even be possible for an electric field to modulate the energy level of the transition state.

Now, let's assume the chemical system has the following asymmetry: the energy level 62 of the left well is higher than the energy level 64 of the right well (FIG. 4C), i.e. the free energy, H₀, of the chemical reaction is not zero.

FIG. 4D shows an example of how an input sinusoidal electric field changes a potential energy surface 66 of a chemical system that has an asymmetry, for example, the one shown in FIG. 4C.

In such a chemical reaction, the population of molecules at a given energy level is governed by a Boltzmann distribution. The energy levels of the molecules at various locations along the Q-axis (and thus, the populations of molecules at those locations along the Q-axis) are subject to thermal fluctuations, which constitute a perturbation to the system. In other words, the system is perturbed by thermal fluctuations.

FIG. 4E shows that, because of the asymmetry (e.g., in this example, H₀ is not equal to zero), the densities of states, the energy levels, and thus, the population distribution profiles of the molecules in the two wells change by different amounts in response to the modulation of the applied sinusoidal electric field. When the population distribution of one of the wells changes (e.g., is driven into non-equilibrium), the time it takes for the distribution to reach equilibrium again (to satisfy Boltzmann statistics) is dependent on the magnitude of the change. As such, at a given frequency of the applied sinusoidal electric field, the two wells may exhibit different responses to the input signal (e.g., different time constants to restore the population distributions in the respective wells back to equilibrium), which may lead to a nonzero relative phase lag (to restore equilibrium in the population distributions) between the two wells. Such a phase lag may have different impacts on the effective barrier heights, on the path lengths 70 and 72 of the forward and reverse paths along the Q dimension, and/or on the average populations of molecules in the two wells.

FIG. 11A shows an example apparatus 73 to implement the system described above. A chemical reaction chamber 74 is operated as a capacitor, with two electrodes 75 on its surface. A sinusoidal electric field is applied across the capacitor plates. Chemical reactants 76 are injected from the left into the reaction chamber and extracted from the chamber from the right as products out 77. An electrical source 78 provides a sinusoidal voltage to be applied across the reaction chamber, and the chamber is coupled in parallel to an electrical inductor 79. The resonant frequency of the capacitor-inductor pair is matched to the frequency of the input electric field. Because of this matching, a significant amount of the applied electrical energy can be recycled in the LC-circuit, and the energy efficiency of the system can be significantly improved.

In FIG. 4F, we plot the results of a simulation, showing the time constant of the forward path (τ_{F}) on the vertical axis (the time constant is related to the average transition time from the left well to the right well, which is also related to the inverse exponent of the effective barrier height for the forward path), as a function of frequency of the applied electric field on the horizontal axis. By selecting the frequency, the effective barrier height of the forward path or the reverse path can be changed, corresponding to a speedup or a slowdown of the transition time along that path. A path time that is made faster can be viewed as a kind of catalysis. A path time that is made slower can be viewed as a kind of de-catalysis (in the literature on conventional catalysis, this effect is sometimes called catalytic poisoning).

In this example system, the input is an electric field, the asymmetry is a non-zero free energy of the chemical reaction (H₀), and the directional effect is an alteration of a time constant for a particular chemical path (e.g., decreasing the time constant, τ, of that path). As such, we call it an E-H₀-τ-asys.

In this example, to achieve catalysis or de-catalysis, the non-directional electric field input is applied over time, and the catalysis or de-catalysis directional effect is achieved along an energy dimension of the system (e.g., by a differential change in the effective barrier height of a particular chemical path). More broadly, this is an example of a very wide range of systems in which a non-directional input and a directional effect comprise a conjugate pair of variables (in this example, time and energy).

We use the phrase “conjugate pair of variables” very broadly to include, for example, any pair of variables of a system that are related to each other in accordance with a principle that governs the system. For example, in Hamiltonian formulations of physics, conjugate variables are coordinates whose Poisson brackets give a Kronecker delta (or a Dirac delta in the case of continuous variables) (e.g., position and momentum, time and energy). In a thermodynamic system, extensive energy transfer can be expressed as the product of a generalized force (in an intensive variable) and a displacement caused by the force (in an extensive variable). Thermodynamic potentials (including, but not limited to, internal energy, Helmholtz free energy, enthalpy, Gibbs free energy, Landau potential) can be expressed as conjugate pairs (including, but not limited to, pressure and volume, temperature and entropy, chemical potential and particle number). A very wide variety of other examples also fall within the phrase “conjugate pair of variables.”

Furthermore, when we use the phrase “a conjugate pair of variables” or “comprising a conjugate pair of variables” or phrases such as “comprising an intensive variable of a conjugate pair of variables”, we mean to include, not only the conjugate pair itself, but also variables that influence and/or are influenced by either or both of the variables of a conjugate pair. Thus, variables that serve as inputs to or outputs from the variables that are technically the conjugate pair are meant to be included in the concept of a conjugate pair of variables, for example, an electric field input that modulates a chemical potential that itself is the intensive variable of a conjugate pair.

In a second example of an asymmetric system, we assume the same electric field input and the same asymmetric potential energy surface for a chemical reaction as in FIG. 4D. At any instant in time, if we take the integrated sum of the forces applied by the electric field at all points along the Q-axis, we also get zero. Therefore, the input is on average non-directional along the Q-axis as well.

In FIG. 4G, we plot the results of a simulation, showing the relative time-averaged population of molecules in the left well in the presence of the input (relative to the time-averaged population in the left well in the absence of the input) on the vertical axis, as a function of frequency of the applied electric field on the horizontal axis. In this simulation, we're using a version of a Fokker-Planck equation to describe the behavior of the system and of the populations of molecules in the two wells and along the Q-axis. We're also assuming a constant temperature and a constant diffusion constant along the Q-axis. For a specific reaction, these parameters may not be constant and/or may have different values than what we've used in our simulations. Again, we can see that at certain frequencies, the average population of molecules in a particular well (e.g., the chemical yield of the reaction) can be changed using the technique that we have described.

In this example, the input is an electric field, the asymmetry is the non-zero free energy of the chemical reaction (H₀), and the directional effect is (in this example) the altered yield of a chemical reaction (e.g., changing the particle number, N, of a particular molecule). As such, we call it an E-H₀-N-asys. And to achieve a directional effect in the form of an altered chemical yield (e.g., a change in the particle number of a particular molecule), a non-directional input is applied that modulates the chemical potentials of the molecules in the system. Then the non-directional input and the directional effect of this example system comprise a conjugate pair of variables, namely, chemical potential and particle number.

In a third example, we also use an asymmetric system having a sinusoidal electric field input, but this time the asymmetry is an off-center location 80 of a transition state along the Q-axis (i.e., Q₀ is not equal to zero along the Q-axis) (FIG. 5A).

FIG. 5B shows an example of how a sinusoidal electric field changes the potential energy surface 82 of a chemical system that has an asymmetry, for example, the one shown in FIG. 5A. Like the example provided in FIG. 4E, because of the asymmetry (e.g., in this example, Q₀ is not at Q=0), the population distribution profiles 84, 86 of the two wells change by different amounts in response to the electric field modulation (FIG. 5C), such that at certain frequencies, the two wells may exhibit different responses to an input.

In FIG. 5D, we plot the results of a simulation, showing the time constant of the forward path (on the vertical axis), as a function of the electric field frequency (on the horizontal axis). As described in the first example, one can catalyze or de-catalyze a particular chemical path in such a chemical system using the techniques that we describe here.

In this example, the input is an electric field, the asymmetry is an off-center location of a transition state (Q₀), and the directional effect is a catalyzing of a particular chemical path, so we call it an E-Q₀-τ-asys. And like the first example provided above, in this example, the non-directional input is applied over time, and the directional effect is achieved along an energy dimension of the system, so the input and effect comprise a conjugate pair of variables, namely, time and energy.

In a fourth example (FIG. 5E), we plot the results of a simulation, showing the relative time-averaged population of molecules in the left well (on the vertical axis), as a function of input frequency (on the horizontal axis). As expected, at certain frequencies, the average population of molecules in a particular well (e.g., the chemical yield of the reaction) can be changed by the techniques that we have described.

In this example, the input is an electric field, the asymmetry is an off-center location of a transition state (Q₀), and the directional effect is the altered yield of a chemical reaction (i.e. particle number, N, of a particular molecule). As such, we call it E-Q₀-N-asys. And similar to the second example above, the non-directional input and the directional effect in this example comprise the conjugate pair of variables of chemical potential and particle number.

In a fifth example, we have an asymmetric system using the asymmetry of the previous example (e.g., Q₀ is not equal to zero, as in FIG. 5A), but this time the input is a force that modulates 88 the location of the transition state Q₀ (e.g., a mechanical force) along the Q-axis (FIG. 6A). Note that in this application, we often refer to any input that modulates the transition state laterally (e.g., moving Q₀ along Q) as a mechanical force, even though strictly speaking, the input that modulates Q₀ need not be mechanical; it could be a wide variety of other inputs. Given a particular system, it may not even be possible for a mechanical force to modulate Q₀. In FIG. 6B, we plot the results of a simulation, showing the normalized population distribution of molecules in both the left well and the right well, as a function of the chemical reaction coordinate (Q-axis), at a fixed input frequency. Again, one can see that in the presence of a modulation, the time-averaged yield of a chemical reaction can be changed.

In this example, the input is a mechanical force, the asymmetry is an off-center location of the transition state (Q₀), and the directional effect is an altered yield of a chemical reaction (e.g., particle number, N, of a particular molecule). As such, we call it m-Q₀-N-asys (we use the small letter m for mechanical, and the capital letter M for magnetic). And again, the non-directional input and the directional effect in this example comprise a conjugate pair of variables of chemical potential and particle number.

In another, sixth, example, a system does not have an intrinsic asymmetry in free energy of the chemical reaction (H₀) or in Q space, i.e., H₀ and Q₀ are equal to zero (FIG. 7A). And there are two inputs: a square wave electric field modulating a potential energy of the transition state, and a square wave mechanical force modulating a location of the transition state. In this example, we assume the two inputs are at the same frequency and that transitions of the electric field lag transitions of the mechanical force by a 90-degree (i.e. τ/2) phase delay (FIG. 7B).

FIG. 7C shows how the two inputs modulate the potential energy surface. The modulation proceeds in a counter-clockwise loop in the energy-Q space, and the existence of a direction of the loop comprises an asymmetry in the system, e.g., the asymmetry is externally applied and the system is now time-variant. A wide variety of other pairs of modulation could be used to provide a loop having a direction. If the mechanical force lagged the electric field, for example, then a clockwise loop would result. And if the modulations of the two inputs were sinusoidal (rather than square) waves, then the loop would be elliptical (rather than rectangular).

FIG. 7D shows the results of a simulation, illustrating the normalized population distribution (on the vertical axis), as a function of the chemical reaction coordinate (Q-axis), at a common frequency of the two inputs, in this case for a counter-clockwise, rectangular loop (e.g., the electric field lags the mechanical force and both are square waves). As in some of the previous examples, the population distribution along the Q-axis, and thus, the time-averaged yield of a chemical reaction, can be changed with such a modulation.

FIG. 11B shows an example apparatus 91 to implement the system described above. As in FIG. 11A, the chemical reaction chamber 93 is treated as a capacitor, with electrodes 95 on its surface to apply an electric field across the capacitor plates. The chemical reactants 97 are injected from the left into the chemical reaction chamber and extracted from the right 99. An electrical source 101 provides the voltage applied across the reaction chamber, and the chamber is coupled in parallel to an electrical inductor 103, with the resonant frequency of the LC-circuit matched to the input frequency such that a significant amount of the electrical energy is circulated and the energy efficiency is improved.

Unlike the example of FIG. 11A, however, an additional second input is provided in the form of a mechanical force 105. If we assume a molecule at a transition state (e.g., an enzyme) is attached to the surface of the reaction chamber, a mechanical force (e.g. a pressure wave, via an acoustic transducer), for example, can be applied to the surface of the reaction chamber. In this example, the frequency of this second input is the same as the frequency of the electric field; however, the electric field lags the mechanical force by a 90-degree (i.e. π/2) phase delay, such that a counter-clockwise modulation is achieved in the energy-Q space.

In this example, there are two inputs, an electrical field and a mechanical force; an asymmetry is externally applied and is the existence of a direction of the applied loop. The directional effect is an altered yield of a chemical reaction (e.g., a particle number, N, of a particular molecule). As such, we call it an m&E-loop-N-asys. In this example, the non-directional inputs and the directional effect can be said to comprise two conjugate pairs of variables: time and energy, and chemical potential and particle number.

In another, seventh, example, we plot the negentropy, i.e. negative entropy, of a system on the vertical axis, rather than its internal energy. For this example, the system comprises a protein, and a higher negentropy implies a more ordered system. In FIG. 8, there are three minima: the one on the left represents an unfolded state of the protein, and the one in the center and the one on the right represent two different folded states of the protein (labeled folded-1 and folded-2). As in the previous example, we apply two inputs to a transition state, in this case, temperature (e.g. via temperature cycling) and surface interaction (e.g. via pH modification). The average of the force applied by each input to the system is zero, i.e., each of the inputs is non-directional. The asymmetry can be the direction of the modulation loop and/or the non-zero entropy difference between the unfolded state and a folded state or between the two folded states. And the directional effect is the pumping or transitioning of the system into a stable state with a higher negentropy, e.g., a more ordered state. In the protein system, this transition may be from the unfolded state to a folded state (the transition on the left in FIG. 8) or vice versa, or from one folded state to another (the transition on the right in FIG. 8) or vice versa. In this example, one of the conjugate pairs of interest may comprise temperature and entropy.

Below, we elaborate on an interpretation of the mechanism of operation of asymmetric systems. And even though the description focuses on manipulation of the internal energy of a system, the same concepts apply to entropy of a system or to another variable of a system that constitutes an extensive variable of a conjugate pair.

Chemical reactions happen on time scales ranging from ˜100 femtoseconds to hours or longer. Theoretical analysis of typical chemical reactions is complicated by a requirement to take into account the time evolution (on the femtosecond time scale) of a large number of nuclear degrees of freedom of a typical molecule/protein on the timescale of the reaction. This problem can be addressed in principle using molecular dynamic (MD) simulations. But current practical limitations of computing power prevent studying more than a microsecond of a reaction.

Theoretical chemists have developed methods that reduce the problem to a time evolution of a single reactive degree of freedom that represents the reaction, while other degrees of freedom are treated as a thermal bath. It is assumed that there is a least energy path along the potential energy surface (PES) from reactants (left well) to products (right well) that passes through a transition state barrier (see FIG. 9A).

We adopt a similar approach here. Note that the dynamics of the single reactive degree of freedom can involve motion of a larger number of atoms depending on the size of the system and, thus, in general, can be represented by a reactive coordinate vector q(t) which characterizes system configuration. For the reactions taking place in the ground electronic state and for large molecules, an approach based on classical mechanics is usually adequate.

To describe a time evolution of a molecular probability density function ρ(q,v,t) one can use the Klein-Kramers equation which is a variant of the Fokker-Plank equation appropriate for the description of the chemical system coupled to a thermal bath:

$$\frac{\partial ρ ( q , v , t )}{\partial t} = [ \begin{matrix}- \frac{\partial }{\partial q} v + \frac{1}{m} \frac{\partial }{\partial v} ( ξ v + \frac{\partial V ( q , t )}{\partial q} ) + \\ \frac{ξ kT}{{m}^{3}} \frac{{\partial }^{2}}{\partial {v}^{2}}\end{matrix} ] ρ ( q , v , t ) ,$$

where V(q,t) is the PES, m is the effective mass along the reactive coordinate of interest (q), ξ is a friction coefficient, T is temperature, k is the Boltzman constant and v is the velocity along q.

This equation describes evolution of the molecular distribution function in the phase space (q, and v). Note, that in the absence of dissipation current, this description incorporates the dynamics described by Hamilton's equations.

If inertial effects can be neglected, then the Klein-Kramers equation simplifies to the Smoluchowski equation in configuration space only:

$$\frac{\partial ρ ( q , t )}{\partial t} = D \frac{{\partial }^{2} ρ ( q , t )}{{\partial }^{2} q} + \frac{D}{kT} \frac{\partial }{\partial q} ( ρ ( q , t ) \frac{\partial U ( q , t )}{\partial q} ) ,$$

where D is the diffusion coefficient which is related to the friction coefficient through the Einstein relation:

$$D = \frac{kT}{ξ} .$$

This equation describes diffusion of the reactive coordinate in the presence of the field of force due to the PES. The absence of the dependence on mass and velocity indicates that the motion described by this equation is overdamped. This equation holds when coupling of the reaction to other degrees of freedom and the bath is strong and, hence, energy dissipation is fast.

Such description is appropriate to describe slow reactions that involve collective reactive motion of a significant number of atoms (e.g., domains of proteins) on up to microsecond time scale because the energy dissipation in the condensed phase happens on femtosecond-picosecond time scale. It may also be appropriate for small molecule reactions on surfaces (e.g., heterogeneous catalysis) if the coupling to the surface is strong.

When the PES is time-independent the steady-state solution of Smoluchowski equation yields the Boltzmann distribution of molecules on the PES which served as an initial distribution for finding time-dependent numerical solutions of the Smoluchowski equation in all simulation results presented below.

Here we are proposing to control the chemical reaction dynamics using externally applied time-dependent perturbations which can modify the PES of the reactive system. These perturbations may involve electrical or mechanical forces. FIGS. 9A, 9B illustrate two situations where an electrical field modulates the transition state energy (FIG. 9A) or a mechanical force modulates the location of the transition state along the reactive coordinate (FIG. 9B). As we will show below such modulations are capable of controlling the direction of chemical reaction and can be use to transduce the energy of the external field into the chemical energy while asymmetry is the determining factor that determines the direction of energy flow.

Considering the thermodynamics of such a system, for an overdamped system, internal energy includes only potential energy as determined by the location of the molecules on the PES. Thus, internal energy is calculated according to:

U(t)=∫ρ(q,t)V(q,t)dq.

If the PES is time dependent, the change in internal energy can be calculated from the First law of thermodynamics:

$$\frac{d U ( t )}{d t} = \dot{W} ( t ) + \dot{Q} ( t ) ,$$

where \dot{W}(t) and \dot{Q}(t) are the rate of work done by an external agent on the system and the heat exchange rate between the system and environment, respectively:

$$\dot{W} ( t ) = \int ρ ( q , t ) \frac{d V ( q , t )}{d t} d q , \\ \dot{Q} ( t ) = \int V ( q , t ) \frac{\partial ρ ( q , t )}{\partial t} d q .$$

Total work done by the external agent (Eᵢₙ) is:

Eᵢₙ=W(t)=∫\dot{W}(t)dt.

Change in the free energy ΔG(t)=ΔU(t)−TΔS(t) can be used as a measure of useful work performed by the external agent (e.g., useful energy stored in the system) because by definition, the free energy represents a maximal amount of work that can be performed by the system. We assume that temperature changes in the system are small because we focus on a system that is strongly coupled to the environment. Typical thermalization times (i.e., cooling) for molecules in solutions are in a picosecond domain; thus our approach is valid up to ˜10 GHz frequency range. For systems that are weakly coupled to the environment (e.g., gas at low pressures), the temperature changes could be taken into account, but we do not consider this case here.

In order to determine free energy changes we need to calculate entropy of the system. Our current treatment relies on classical mechanics therefore we use Shannon's definition of the entropy as applied to continuous configuration space determined by our model:

S(t)=−k∫ρ(q,t)ln [ρ(q,t)]dq.

Finally, efficiency is defined as:

$$Efficiency ( t ) = \frac{Δ G ( t )}{{E}_{ i n} ( t )} .$$

Yield is defined as a relative change in the number of molecules in the left well (the reactant well).

FIG. 9C shows the simulated population of molecules in the left well after the thermally equilibrated system has been exposed to the sinusoidal modulation of the transition state barrier height (ΔG_{f}) as a single modulated parameter. The data indicate that there is change in relative population of the left well induced by the modulation. At first it might be hard to see why population flows from one well to another since modulation of the transition state energy (i.e., of the reaction barrier ΔG_{f}) should only affect reaction rates but not relative populations in the wells. The reason for that is the effect that modulation has on the density of states available in the left and right wells. Depending on the asymmetry of the PES, change in the transition state energy differently affects the number of accessible energy states in the left and right wells which induces population flow between wells. In this case asymmetry can be introduced into the PES by the nonzero value of the H₀ (e.g., the driving force, FIG. 9A) and/or a nonzero value of Δq (displacement of the transition state from the center between two wells, FIG. 9B). Both PES asymmetries result in pumping action between the wells.

Asymmetry can also be introduced into the system by appropriate choice of the relative phase between two external fields applied to the system as shown in FIG. 9D. In this case PES of the system can be completely symmetric. FIG. 9E shows relative population of the left well in response to modulation by two fields (e.g., electrical and mechanical). The phase between two modulations was fixed to +π/2 or −π/2 resulting in counter-clockwise or clockwise modulation trajectory. Simulation data show that, as a result of such two-parameter modulation, the population is transferred to the left or right wells depending on the direction of modulation trajectory. Note, that for counter-clockwise trajectory, molecules are pumped into the left well against the driving force H₀. This amounts to transduction of the energy of the external agent (which is causing the modulation) into chemical energy. Next, we explored the efficiency of such energy transduction under various conditions.

FIG. 9F shows the modulation frequency dependence of the thermodynamic efficiency and the yield for different reaction barrier heights (ΔG_{f}); efficiency and yield values were calculated after the first modulation period (i.e. after completion of one trajectory run around the circle). Note, that there are two peaks in the frequency dependence of efficiency. The lower frequency peak is due to a transfer of molecules to the left well since it appears at similar frequency as a corresponding peak in the yield (see the right graph in FIG. 9F). Thus, the peak at lower frequencies describes transduction of modulation energy into chemical energy. Note, that lower frequency peak appears at a frequency roughly equal to the inverse transition time over the reaction barrier, i.e., its location on the frequency scale is determined by the reaction time. Reaction time depends on barrier height and temperature, hence the clear shift of peak location with the barrier height (FIG. 9F). The higher frequency peak in the efficiency dependence is due to heating of the system by the modulating fields which is expected when modulation frequency becomes comparable to intrawell equilibration/dissipation time; such non-adiabatic perturbations are associated with entropy production and energy loss.

FIG. 9G shows the modulation frequency dependence of the thermodynamic efficiency and the yield for different values of driving force (H₀). Simulation data indicate that transduction efficiency decreases for larger opposing forces (i.e. larger H₀), which is as expected because the load is expected to reduce efficiency.

The inherently higher efficiency and yield of the two parameter modulation can be understood from the following simple picture based on the analysis of the population dynamics along the circular or rectangular trajectory. When the system is moved along the trajectory from the entry point A (see FIG. 9D), the barrier height is low, thus molecules move from the right to the left well relatively fast (due to increasing number of available energy states in the left well), however, when the system continues from the point B to point A, the barrier height is higher thus preventing molecules from escaping back into the right well. As a result after completion of a single counter clock trajectory cycle, population of molecules in the left well is increased.

Simulations presented in FIGS. 9G, 9H were performed assuming starting initial condition corresponding to the modulation trajectory entry point A (see FIG. 9D). It is implied that molecules are first adiabatically and reversibly brought to the starting point A from an unperturbed state (i.e., q=0); the slow entry and subsequent extraction guarantees no additional energy losses.

Presented simulations were performed for specified sets of parameters only. Optimization of all the above parameters should further increase the performance. For example FIG. 9H shows that thermodynamic transduction efficiencies of ˜75% can be achieved using larger modulation amplitudes.

There are multiple ways that transduction efficiency and yield could be further improved. For example instead of sinusoidal modulation (resulting in circular trajectory as shown in FIG. 9D), a rectangular trajectory could be used with each modulation leg corresponding to change in either the δ or Δq at different rate. FIG. 9I shows preliminary comparison of transduction efficiency for circular and rectangular trajectories; these simulation data suggest that rectangular modulation enables higher efficiencies.

Even higher performance is expected for slower reactions since the transduction efficiency peak appears at lower frequencies for larger reaction barriers (FIG. 9F), i.e., the separation between the lower frequency peak (useful transduction) and the higher frequency peak (heat loss) is larger. This makes modulation at lower frequencies more adiabatic and reversible, leading to higher transduction efficiency.

The above description focused on chemical reactions and their dynamics to provide some insight into how ASYS operates and how it alters the internal energy of objects and/or systems. Another example of ASYS can be provided from biology, and this time, with the negentropy of a system and/or an object altered instead. Negentropy is the negative entropy, and is used as a measure of the level of order or complexity. In a self-organizing system, the interactions between sub-systems and/or objects occur such that the overall interaction energy is lowered, which then compensates an increase in the negentropy of a system to a more ordered state (e.g. protein folding, homeostasis). ASYS can be applied to such a system to manipulate its negentropy and alter its level of self-organization, and/or it can enable self-organization to occur in a system whose interaction energy would not otherwise lead to an increase in its negentropy, i.e., it would not self-organize in the absence of ASYS transducing an external energy towards the system's negentropy.

A convenient analogy to better describe the key concepts of our ASYS technique employs the basic concepts and equations that are used in Noether's theorem. Noether's theorem relates symmetries to conservation laws, with the two variables comprising a conjugate pair. For example, a system that is symmetric in time (e.g., that is invariant in time) leads to conservation of energy. Similarly, a system that is invariant in space (e.g., symmetric in position) leads to conservation of momentum, and so on.

The ASYS technique can be regarded as the inverse Noether's theorem, where the absence or violation of a symmetry (e.g., an asymmetry) leads to the non-conservation of another variable (e.g., a directional effect) with the input and output variables comprising a conjugate pair. In the example of yield manipulation in a chemical reaction provided above, there may be an asymmetry in chemical potential, which leads to a directional change in particle number (of a certain molecule), with chemical potential and particle number comprising a conjugate pair. Furthermore, sets of equations similar to those employed in Noether's theorem can also be used to optimize and quantify the ASYS technique for a particular asymmetric system, simply by assuming that a certain symmetry does not apply and then looking at how the variable to be conserved (or not) gets impacted and by how much.

One can interact (we use the term interact in a similar very broad sense as the term interaction, as explained earlier) with such a system (as described above) by, for example, using or causing the system to achieve the directional effect (including, but not limited to, a net yield change in a chemical reaction) from a non-directional input (including, but not limited to, a sinusoidal electric field), based on an asymmetry of the system (including, but not limited to, a nonzero free energy of the reaction).

The interaction may also include any apparatus that embodies such a system.

As shown in the flowchart of FIG. 10A, interaction with the system may involve identifying, selecting, or using a desired or intended directional effect 90 to be achieved or obtained or caused in a given system as a basis for designing a system for a certain functionality or performance or both. In some examples, the design can proceed by identifying a desired directional effect 90, choosing an appropriate system for that effect 92, choosing a suitable extensive variable of a conjugate pair that relates to the effect 94, identifying a corresponding intensive variable of the conjugate pair 96, identifying an input that relates to the intensive variable 98, identifying a suitable asymmetry that couples the input and output 100, choosing a suitable way to apply the input in a non-directional manner 102, simulating the system to quantify its performance, aspects, and features 104, and implementing the resulting system 106. For example, if a desired directional effect is a higher yield of a chemical reaction, one can choose an appropriate conjugate pair of variables (including, but not limited to, time and energy). In that example, the desired outcome is a directional effect along the energy variable. A suitable asymmetry in the system is identified (including, but not limited to, a nonzero free energy difference between the substrate and the product molecules). Then the design process includes identifying and implementing an appropriate non-directional input along the time variable, such that the asymmetry in the perturbed system will translate it to the directional effect of interest.

As shown in FIG. 10B, one can interact with a system by manipulating it. The steps of the manipulating can include the following. A desired manipulation is identified 110. This could be for example, implementing a new function, for example, filtering. Given the system and the directional effect, how the directional effect needs to be altered would be identified 112. Given the non-directional input and the asymmetry, the appropriate signal to use for implementing the new function would be identified 114. Given the system response, an appropriate step to implement the new function would be chosen 116. Given how the directional effect needs to be altered, how the input and/or the asymmetry needs to be altered would be simulated 118. A suitable way would be identified to alter the input and/or the asymmetry accordingly 120. The system would be simulated to ensure the new function is implemented within acceptable specifications 122 And the new function would be implemented 124.

This manipulation may involve, for example, analyzing (including, but not limited to, magnitude of the yield change as a function of frequency) or optimizing the system (including, but not limited to, adjusting the frequency and/or the phase of two inputs relative to each other, to maximize the directional effect). The system may be optimized for work done, for energy efficiency, for operation in a desired regime, and/or for a particular load. This manipulation may also involve implementing a new function in the system. This function may be filtering (including, but not limited to, change the system response to an input at certain frequencies), adaptive filtering, compression, de-compression, sampling, de-sampling, feature extraction, spectrum analysis, storage or modulation.

FIG. 10C shows an example embodiment to implement such interaction in the form of design and/or manipulation.

A user 130 can identify or plan a desired interaction with an asymmetric system 132, and can plan and execute a physical embodiment 134 based on a design or manipulation. A computer 136 can be used. Signal processing software 138 and simulation software 140 are run by a processor 142 that has access to storage 144 as needed. The result of the computer-implemented process is a plan of how to achieve the desired interaction with the asymmetric system 146. Any of a wide variety of computer and software platforms can be used to implement the concepts described here.

For a given input and directional effect of a system, interaction can include, for example, signal processing concepts and methodologies. We refer to “signal processing” very broadly to include, but not be limited to, analysis, interpretation, and/or manipulation of signals. We can use signal processing to describe and/or to interpret any aspect or feature of a system and any combination of them.

We can also use signal processing to transform any such signal. We use the term transform very broadly. Without limitation, examples of transforms include an integral transform (including, but not limited to, Abel, Fourier, Short-time Fourier, Hankel, Hartley, Hilbert, Hilbert-Schmidt integral operator, Laplace, Inverse Laplace, Two-sided Laplace, Inverse two-sided Laplace, Laplace-Stieltjes, Linear canonical, Mellin, Inverse Mellin, Poisson-Mellin-Newton cycle, Radon, Stieltjes, Sumudu, Wavelet), a discrete transform (including, but not limited to, Binomial, Discrete Fourier, Fast Fourier, Discrete cosine, Modified discrete cosine, Discrete Hartley, Discrete sine, Hankel, the determinant of the Hankel matrix, Irrational base discrete weighted, Number-theoretic, Stirling, Z-transform), a data-dependent transform (including, but not limited to, Karhunen-Loéve), or another transform (Bäcklund, Bilinear, Box-Muller, Burrows-Wheeler, Wavelet, Distance, Fractal, Hadamard, Hough, Legendre, Möbius, Perspective, Y-delta). The transform may be a one-variable transform or a multi-variable transform. The signal processing may be used to effect more than one type of transform, including combinations and sequences of transforms and transforms that may be developed in the future.

The system may very broadly be any kind of system, including, for example, a physical system, a chemical system, a biological system, a social system, an economic system, or another system, or a combination of any two or more of such systems (including, but not limited to, a biochemical system or a biophysical system).

The chemical system may comprise a chemical reaction. The chemical reaction may comprise an intermediate chemical reaction. The chemical reaction may comprise a surface reaction, a bulk reaction, or a membrane reaction, or combinations of them. The chemical reaction may comprise an organic reaction or an inorganic reaction or a combination of the two. The chemical reaction may comprise an enzymatic reaction, a catalytic reaction, a non-catalytic reaction, or combinations of them. The chemical reaction may comprise a spontaneous reaction or a non-spontaneous reaction, or a combination of the two. The chemical reaction may comprise an exothermic reaction or an endothermic reaction, or a combination of the two. The chemical reaction may comprise a single chemical path or multiple possible chemical paths.

The conjugate pair of variables of interest in the system may be very broadly any conjugate pair, for example, position and momentum, time and energy, temperature and entropy, pressure and volume, electric field and polarizability, magnetic field and magnetization, stress and strain, rotation angle and angular momentum, chemical potential and particle number, electric potential and electromotive force, two orthogonal polarization vectors of an electromagnetic beam, surface are and surface tension, or another conjugate pair. There may be more than one conjugate pair of variables in the system and combinations of them that are of interest.

The system may be a feedback system. The system may be time-invariant or time-variant. It may be linear or nonlinear. The system may be a continuous-time system or a discrete-time system. The system may include a combination of such systems.

The non-directional input may comprise an intensive variable of a conjugate pair of variables.

The non-directional input may be a signal. It may be externally applied or may be intrinsic to the system. It may be an input signal, a noise signal, a control signal, or an intermediate signal. We intend the term intermediate to broadly refer to, for example, any signal that is not an external input signal into the system or an output signal out of the system. The non-directional input may be time-independent or time-dependent. It may be continuous-time or discrete-time. It may be deterministic or stochastic. There may be more than one input and any combination of them.

The non-directional input may be an influence.

An influence may be chemical, electrical, magnetic, thermal, electromagnetic, flow, pressure, mechanical, gravitational, or another influence. It may be a combination of two or more of the above mentioned influences. If two or more, the influences may be of the same type of influence or of at least two different types of influences. If two or more, the influences may have the same phase. They may also have different phases with a fixed relationship or different phases with a varying relationship. If two or more, the influences may have the same frequency. They may also have different frequencies with a fixed relationship or different frequencies with a varying relationship.

The asymmetry may be an absence or a violation of a non-isometric symmetry, a directional symmetry, a reflection symmetry, a rotation symmetry, a translational symmetry, a glide reflection symmetry, a rotoreflection symmetry, a helical symmetry, a scale symmetry, or a combination of two or more of the above mentioned symmetries.

The asymmetry may be externally applied or it may be intrinsic to the system. The asymmetry may be time-independent or time-dependent. It may be a one-variable asymmetry or a multi-variable symmetry. There may be more than one asymmetry in the system. If two or more, the asymmetries may be an absence or a violation of the same type of symmetry or antisymmetry, or of at least two different types of symmetries or antisymmetries.

The directional effect may comprise an extensive variable of a conjugate pair of variables.

The directional effect may be an output signal, a noise signal, a control signal, or an intermediate signal. It may be time-independent or time-dependent. It may be continuous-time or discrete-time. It may be deterministic or stochastic. There may be more than one directional effect achieved. If two or more, the directional effects may be in the same variable or they may be in at least two different types of variables.

The directional effect may include doing mechanical work. The mechanical work may be altering the kinetic energy of a system. The directional effect may include altering the potential energy of a system. The potential energy may be gravitational potential energy, elastic potential energy, chemical potential energy, electrical potential energy (e.g. electrostatic, electrodynamic or magnetic, nuclear), thermal potential energy, and/or rest mass energy. The directional effect may include doing thermodynamic work. Thermodynamic work may include altering enthalpy and/or entropy of a system and/or doing pressure-volume work on the system. The directional effect may be doing organizational work. The organizational work may be altering the order, complexity, pattern, structure, emergent property, and/or behavior of a system.

Implementation of the vertical and lateral modulation of the chemical PES relies on the ability to impose modulation of the binding interactions between atoms of the molecule. This can be achieved in multiple ways. First external fields can be used that directly interact with the electronic states of the molecule, thereby affecting energy of the selective configuration of the molecule (FIG. 11C). For example, frequently the transition state of the reacting molecule exhibits partial charge separation which is characterized by a dipole moment. Interaction of such dipole moment with the external electric field will directly affect the energy of the transition state enabling vertical modulation (and may be also lateral modulation) of the PES. In this case, the time profile of the electric field can have an arbitrary time profile allowing us to impose any desired vertical modulation profile upon the system.

Secondly, the modulation of the PES can be achieved by imposing modulation of the properties of the environment in which reacting molecules reside. The environment can include, for example, any other molecules/atoms in the surrounding area that are interacting with the reacting molecule. For example if the reacting molecule is the substrate of an enzyme, then the amino residues of the enzyme in the active pocket will interact with the reactive molecule and have a major effect on the transition state of the reactant enabling catalysis. Any external perturbation of the enzyme that results in changes of the geometry of the active pocket will modulate the PES of the reactant. In turn, the perturbation of the enzyme structure can be achieved in multiple ways, e.g., electric fields, acoustic fields, pressure, changes in pH, temperature, ionic strength or specific ligands; furthermore enzymes can be derivatized with external force “mediators” (or “influence mediators”) such as charged or magnetic beads or linkers that couple the externally applied force (e.g. electric or magnetic field or mechanical force) to the enzyme and thereby inducing changes in the structure of the active pocket.

Generally any supramolecular structure can be used to build an active environment 201 around the reactant 202, e.g., aptamers 204 or molecules/materials 206 with nanocavities (e.g. zeolites, polymers, cyclodextrins, carbon nanotubes and related) can be employed for this purpose (FIGS. 11F, 11G). In such active pockets, both vertical and lateral modulation of the transition state energy and location is possible by using external fields/forces and attached influence modulators, such as charged/magnetic beads 208 and directly attached linkers 210 exposed to mechanical force. In particular, the lateral modulation (i.e., modulation of transition state location along the chemical reaction coordinate) can be achieved by fine tuning the geometry of the active pocket in such a way that differently affects interaction strength with the reactive molecule configurations, which are structurally closer to reactants or products. The methods to design such an active pocket may involve combinations of ab initio simulations, catalytic antibodies against corresponding transition state analogs, in vitro evolution, and other methods and processes.

The above approach enables selection of different external forces for induction of vertical or lateral modulation (e.g. electrical and mechanic), thereby allowing simultaneous modulation of multiple parameters. In order to achieve stronger electric field strengths, an enzyme 214 may be linked to a surface 216 of a reaction chamber 218 where formation of electrical double layer can be used for field enhancement (e.g., FIG. 11C). It should be noted that even small deformation of the supramolecular environment (e.g. enzyme) of the reactant can lead to large changes in the transition state energy, e.g., the supramolecular environment can also be used to amplify and enhance the effect of external perturbation on the chemical reactant PES.

Similar approaches as above can be used for surface catalyzed reactions, which are common in energy and chemical manufacturing industries. The energy and location of the transition state for surface catalyzed reactions is sensitive to the details of coordinating interactions between the molecule and the surface catalyst. Deformation of the surface (e.g., compression or stretching) can lead to changes in the structure of the surface and are expected to affect both the energy and location of the transition state. For example, an electric field can be used to modulate the energy of the transition state while a mechanical deformation (e.g. by direct stretching/compression or by acoustic surface waves or using a piezoelectric substrate, 226, FIG. 11E) of the surface catalyst may enable lateral modulation; combinations of both will enable simultaneous two-parameter modulation (e.g., FIG. 11D). To enhance the effect of mechanical deformation, the surface catalyst 220 may be coated on a surface 222 of a flexible substrate 224, e.g. plastic, silicon resins or similar (FIG. 11E).

A wide variety of physical implementations of an ASYS system are possible. Examples have been provided earlier. In general, the interaction with an ASYS system may comprise an apparatus with a site for a reaction and a device interacting with the system. The reaction may comprise a chemical reaction and/or a biochemical reaction and a wide variety of other possible reactions. The device may comprise one or more controlled inputs, such as voltage, mechanical force, temperature, and/or pressure, or others or combinations of them. The device may also comprise a surface on which or near where the reaction takes place.

The directional effect may comprise, for example, converting a type of non-chemical energy into another non-chemical energy. Electrical energy may be converted into rotary power or mechanical work, for example. Rotary power or mechanical work may be converted into electrical energy, for example.

Other examples are possible.

For example, the directional effect may comprise converting a type of chemical energy into a non-chemical energy. A chemical fuel energy may be converted into a non-chemical energy. A chemical energy may be converted into electrical energy or rotary power or mechanical work.

The directional effect may comprise converting a type of non-chemical energy into a chemical energy. Electrical energy may be converted into a chemical energy. A non-chemical energy may be converted into a high energy density chemical fuel (e.g. methane, ethane, or hydrogen), into a biofuel (e.g. methanol or ethanol), or it may drive a chemical fuel process (e.g. gasoline cracking, or gasoline synthesis).

The directional effect may comprise converting a type of chemical energy into another type of chemical energy. The chemical reaction may comprise CO₂ reduction, glucose to fructose conversion, or ethylene production.

The direction effect may comprise manipulating a chemical reaction. The chemical reaction may be an intermediate chemical reaction. For example, the reaction for the combustion of methane is: CH₄+2O₂→CO₂+2H₂+heat; with intermediate reactions given as: CH₄+2O₂→CO+H₂+H₂O, 2CO+O₂→2CO₂, and 2H₂+O₂→2H₂O.

The directional effect, in this example, may comprise running this reaction in reverse, i.e. CO₂+2H₂O→CH₄+2O₂, providing the necessary energy by transducing it from an externally applied input, e.g. electrical energy (FIG. 12A). The chemical reaction may also comprise one or more of the intermediate reactions here, e.g. 2CO₂→2CO+O₂, 2H₂O→2H₂+O₂ (FIG. 12B), and/or CO+H₂+H₂O→CH₄+2O₂.

A wide variety of kinds of manipulation are possible. For example, the manipulation may comprise controlling a direction of the reaction. It may comprise altering a final substrate and/or a final product concentration or the ratio of the two concentrations. It may comprise doing work on the system that the system would otherwise not do, including against or along other influences and/or gradients. The manipulation may further comprise catalyzing the reaction. And/or it may comprise specific enhancement and/or suppression of reactions and/or chemical paths. The manipulation may also comprise increasing, decreasing, or reversing a spontaneity of the reaction. It may comprise changing a probability of a specific path and/or product, relative to another alternative path or product, to change a yield of the specific path and/or product.

A result of the method when used in chemical reactions or pathways comprises new mixtures and/or products.

The system may be used in chemical manufacturing, industrial processing, catalysis, chemical fuel production, electricity generation, rotary power or mechanical work generation, energy storage, and/or reduction of undesired chemicals (e.g. greenhouse gases).

The directional effect may comprise altering the negentropy (i.e., negative entropy) of a system. The system may comprise a self-organizing system, such as a protein or a self-assembling molecule. It may comprise a process, such as cell signaling, homeostasis, or developmental stages of a cell or a living organism (e.g. reproduction, growth, differentiation, death). The system may be at the molecular, cellular or behavioral scale. It may be used in basic life science research, medicine (e.g. discovery, treatment, or monitoring), and/or synthetic life processes and products.

The directional effect may comprise transporting an object. The object may be transported against an opposing force and/or gradient. The object may comprise a micro-object, such as an ion, a molecule, a biomolecule, and/or a biological cell, or it may comprise a macro-object, such as a transportation vehicle. The system may be used in mechanics, biological transportation, chemical transportation, and/or vehicular transportation.

The directional effect may comprise altering the property of an object and/or a process. The property may comprise structure, complexity, strength, elasticity, and/or weight. The system may be used in material science or manufacturing.

The directional effect may comprise altering the electromagnetic property of an object and/or a process. The system may be used in electronics or communications.

In addition to and based on all of the above, and combinations of them, other examples may also be included. For example, an electric field input may be applied to an asymmetric system, in which a spontaneous chemical reaction may also be an input, and the energy released from the reaction may be converted into electrical energy and extracted out of the system (e.g., the output electrical energy would be the directional effect). Another example is a similar asymmetric system, in which the external influence may be a magnetic field, rather than an electric field.

The techniques described here could be used to modify or improve existing asymmetric systems and/or the interactions with such systems. For example, a Brownian ratchet may be designed and/or manipulated using signal processing.

Many other systems can be interacted with, based on a wide variety of combinations of non-directional inputs, asymmetries, directional effects, systems, and conjugate pairs of variables.

Other implementations are also within the scope of the following claims.

1. A combination comprising
interaction with a system that has a perturbation,
in such perturbed system a non-directional input is applied to a first variable of the system, and
based on an asymmetry of the perturbed system, achieving a directional effect in a second variable of the system,
the first and second variables comprising a conjugate pair of variables,
and in which at least one of the following pertains:
the interaction occurs other than by an apparatus and other than in a way that actually achieves the directional effect, or
the conjugate pair is other than position and momentum, or
the input or the asymmetry is in a dimension other than spatial coordinates, or
the directional effect is other than one-dimensional translational motion and other than one-dimensional rotary motion.

2. The combination of claim 1 in which the interaction comprises causing the system to actually achieve the directional effect.

3. The combination of claim 1 in which the interaction includes an apparatus.

4. The combination of claim 1 in which the interaction comprises designing the system.

5. The combination of claim 1 in which the interaction comprises manipulating the system.

6. The combination of claim 5 in which the manipulating comprises analyzing the system.

7. The combination of claim 5 in which the manipulating comprises optimizing the system.

8. The combination of claim 7 in which the system is optimized for work done.

9. The combination of claim 7 in which the system is optimized for energy efficiency.

10. The combination of claim 7 in which the system is optimized for operation in a desired regime.

11. The combination of claim 7 in which the system is optimized for a particular load.

12. The combination of claim 5 in which the manipulation comprises implementing a function in the system.

13. The combination of claim 12 in which the function comprises filtering.

14. The combination of claim 12 in which the function comprises adaptive filtering.

15. The combination of claim 12 in which the function comprises compression.

16. The combination of claim 12 in which the function comprises de-compression.

17. The combination of claim 12 in which the function comprises sampling.

18. The combination of claim 12 in which the function comprises de-sampling.

19. The combination of claim 12 in which the function comprises feature extraction.

20. The combination of claim 12 in which the function comprises spectrum analysis.

21. The combination of claim 12 in which the function comprises storage.

22. The combination of claim 12 in which the function comprises modulation.

23. The combination of claim 1 in which the interaction is based on signal processing.

24. The combination of claim 23 in which the signal processing comprises description of an aspect of the system.

25. The combination of claim 23 in which signal processing comprises interpretation of an aspect of the system.

26. The combination of claim 23 in which the signal processing comprises taking the transform of a variable.

27. The combination of claim 26 in which the transform comprises an integral transform.

28. The combination of claim 27 in which the integral transform comprises a Fourier transform.

29. The combination of claim 27 in which the integral transform comprises a Laplace transform.

30. The combination of claim 27 in which the integral transform comprises a wavelet transform.

31. The combination of claim 27 in which the integral transform comprises a Hilbert transform.

32. The combination of claim 27 in which the transform comprises a discrete transform.

33. The combination of claim 32 in which the discrete transform comprises a binomial transform.

34. The combination of claim 32 in which the discrete transform comprises a discrete Fourier transform.

35. The combination of claim 32 in which the discrete transform comprises a fast Fourier transform.

36. The combination of claim 32 in which the discrete transform comprises a Z-transform.

37. The combination of claim 26 in which the transform comprises a data-dependent transform.

38. The combination of claim 26 in which the transform comprises a transform other than an integral, discrete or data-dependent transform.

39. The combination of claim 26 in which the transform comprises a one-variable transform.

40. The combination of claim 26 in which the transform comprises a multi-variable transform.

41. The combination of claim 1 in which the system comprises a physical system.

42. The combination of claim 1 in which the system comprises a chemical system.

43. The combination of claim 42 in which the chemical system comprises a chemical reaction.

44. The combination of claim 43 in which the chemical reaction comprises an intermediate chemical reaction.

45. The combination of claim 43 in which the chemical reaction comprises a surface reaction.

46. The combination of claim 43 in which the chemical reaction comprises a bulk reaction.

47. The combination of claim 43 in which the chemical reaction comprises a membrane reaction.

48. The combination of claim 43 in which the chemical reaction comprises an organic reaction.

49. The combination of claim 43 in which the chemical reaction comprises an inorganic reaction.

50. The combination of claim 43 in which the chemical reaction comprises an enzymatic reaction.

51. The combination of claim 43 in which the chemical reaction comprises a catalytic reaction.

52. The combination of claim 43 in which the chemical reaction comprises a non-catalytic reaction.

53. The combination of claim 43 in which the chemical reaction comprises a spontaneous reaction.

54. The combination of claim 43 in which the chemical reaction comprises a non-spontaneous reaction.

55. The combination of claim 43 in which the chemical reaction comprises an exothermic reaction.

56. The combination of claim 43 in which the chemical reaction comprises an endothermic reaction.

57. The combination of claim 43 in which the chemical reaction comprises a single chemical path.

58. The combination of claim 43 in which the chemical reaction comprises multiple possible chemical paths.

59. The combination of claim 1 in which the system comprises a biological system.

60. The combination of claim 1 in which the system comprises a social system.

61. The combination of claim 1 in which the system comprises an economic system.

62. The combination of claim 1 in which the system comprises a combination of two or more of physical, chemical, biological, social and economic systems.

63. The combination of claim 1 in which the conjugate pair of variables comprises position and momentum.

64. The combination of claim 1 in which the conjugate pair of variables comprises time and energy.

65. The combination of claim 1 in which the conjugate pair of variables comprises temperature and entropy.

66. The combination of claim 1 in which the conjugate pair of variables comprises pressure and volume.

67. The combination of claim 1 in which the conjugate pair of variables comprises electric field and polarizability.

68. The combination of claim 1 in which the conjugate pair of variables comprises magnetic field and magnetization.

69. The combination of claim 1 in which the conjugate pair of variables comprises stress and strain.

70. The combination of claim 1 in which the conjugate pair of variables comprises rotation angle and angular momentum.

71. The combination of claim 1 in which the conjugate pair of variables comprises chemical potential and particle number.

72. The combination of claim 1 in which the conjugate pair of variables comprises electric potential and electromotive force.

73. The combination of claim 1 in which the conjugate pair of variables comprises two orthogonal polarization vectors of an electromagnetic beam.

74. The combination of claim 1 in which the conjugate pair of variables comprises surface area and surface tension.

75. The combination of claim 1 in which there are two or more conjugate pairs of variables in the system.

76. The combination of claim 1 in which the system comprises a feedback system.

77. The combination of claim 1 in which the system comprises a time-invariant system.

78. The combination of claim 1 in which the system comprises a time-variant system.

79. The combination of claim 1 in which the system comprises a linear system.

80. The combination of claim 1 in which the system comprises a nonlinear system.

81. The combination of claim 1 in which the system comprises a continuous-time system.

82. The combination of claim 1 in which the system comprises a discrete-time system.

83. The combination of claim 1 in which the non-directional input comprises an intensive variable of a conjugate pair of variables.

84. The combination of claim 1 in which the non-directional input comprises a signal.

85. The combination of claim 84 in which the non-directional input comprises an externally applied signal.

86. The combination of claim 84 in which the non-directional input comprises a signal that is intrinsic to the system.

87. The combination of claim 84 in which the non-directional input comprises an input signal.

88. The combination of claim 84 in which the non-directional input comprises a noise signal.

89. The combination of claim 84 in which the non-directional input comprises a control signal.

90. The combination of claim 84 in which the non-directional input comprises an intermediate signal.

91. The combination of claim 84 in which the non-directional input comprises a time-independent signal.

92. The combination of claim 84 in which the non-directional input comprises a time-dependent signal.

93. The combination of claim 84 in which the non-directional input comprises a continuous-time signal.

94. The combination of claim 84 in which the non-directional input comprises a discrete-time signal.

95. The combination of claim 84 in which the non-directional input comprises a deterministic signal.

96. The combination of claim 84 in which the non-directional input comprises a stochastic signal.

97. The combination of claim 1 in which the non-directional input comprises more than one signal.

98. The combination of claim 1 in which the non-directional input comprises an influence.

99. The combination of claim 98 in which the non-directional input comprises a chemical influence.

100. The combination of claim 98 in which the non-directional input comprises an electrical influence.

101. The combination of claim 98 in which the non-directional input comprises a magnetic influence.

102. The combination of claim 98 in which the non-directional input comprises a thermal influence.

103. The combination of claim 98 in which the non-directional input comprises an electromagnetic influence.

104. The combination of claim 98 in which the non-directional input comprises a flow influence.

105. The combination of claim 98 in which the non-directional input comprises a pressure influence.

106. The combination of claim 98 in which the non-directional input comprises a mechanical influence.

107. The combination of claim 98 in which the non-directional input comprises a gravitational influence.

108. The combination of claim 98 in which the non-directional input comprises a combination of two or more influences.

109. The combination of claim 108 in which the influences are of the same type of influence.

110. The combination of claim 108 in which the influences are of at least two different types of influences.

111. The combination of claim 108 in which the influences have the same phase.

112. The combination of claim 108 in which the influences have different phases with a fixed relationship.

113. The combination of claim 108 in which the influences have different phases with a varying relationship.

114. The combination of claim 108 in which the influences have the same frequency.

115. The combination of claim 108 in which the influences have different frequencies with a fixed relationship.

116. The combination of claim 108 in which the influences have different frequencies with a varying relationship.

117. The combination of claim 1 in which the asymmetry comprises an absence or a violation of a non-isometric symmetry.

118. The combination of claim 1 in which the asymmetry comprises an absence or a violation of a directional symmetry.

119. The combination of claim 1 in which the asymmetry comprises an absence or a violation of a reflection symmetry.

120. The combination of claim 1 in which the asymmetry comprises an absence or a violation of a rotational symmetry.

121. The combination of claim 1 in which the asymmetry comprises an absence or a violation of a translational symmetry.

122. The combination of claim 1 in which the asymmetry comprises an absence or a violation of a glide reflection symmetry.

123. The combination of claim 1 in which the asymmetry comprises an absence or a violation of a rotoreflection symmetry.

124. The combination of claim 1 in which the asymmetry comprises an absence or a violation of a helical symmetry.

125. The combination of claim 1 in which the asymmetry comprises an absence or a violation of a scale symmetry.

126. The combination of claim 1 in which the asymmetry comprises an absence or a violation of two or more symmetries.

127. The combination of claim 1 in which the asymmetry comprises an externally applied asymmetry.

128. The combination of claim 1 in which the asymmetry comprises an asymmetry that is intrinsic to the system.

129. The combination of claim 1 in which the asymmetry comprises a time-independent asymmetry.

130. The combination of claim 1 in which the asymmetry comprises a time-dependent asymmetry.

131. The combination of claim 1 in which the asymmetry comprises a one-variable asymmetry.

132. The combination of claim 1 in which the asymmetry comprises a multi-variable asymmetry.

133. The combination of claim 1 in which there is more than one asymmetry.

134. The combination of claim 133 in which all the asymmetries comprise an absence or a violation of the same type of symmetry or antisymmetry.

135. The combination of claim 133 in which the asymmetries comprise an absence or a violation of two or more types of symmetries or antisymmetries.

136. The combination of claim 1 in which the directional effect comprises an extensive variable of the conjugate pair of variables.

137. The combination of claim 1 in which the directional effect comprises an output signal.

138. The combination of claim 1 in which the directional effect comprises a noise signal.

139. The combination of claim 1 in which the directional effect comprises a control signal.

140. The combination of claim 1 in which the directional effect comprises an intermediate signal.

141. The combination of claim 1 in which the directional effect comprises a time-independent signal.

142. The combination of claim 1 in which the directional effect comprises a time-dependent signal.

143. The combination of claim 1 in which the directional effect comprises a continuous-time signal.

144. The combination of claim 1 in which the directional effect comprises a discrete-time signal.

145. The combination of claim 1 in which the directional effect comprises a deterministic signal.

146. The combination of claim 1 in which the directional effect comprises a stochastic signal.

147. The combination of claim 1 in which more than one directional effect is achieved.

148. The combination of claim 147 in which the directional effects are in the same type of variable.

149. The combination of claim 147 in which the directional effects are in at least two different types of variables.

150. The combination of claim 1 in which the directional effect comprises doing mechanical work.

151. The combination of claim 150 in which doing mechanical work comprises altering the kinetic energy of a system.

152. The combination of claim 1 in which the directional effect comprises altering the potential energy of a system.

153. The combination of claim 152 in which the potential energy comprises gravitational potential energy.

154. The combination of claim 152 in which the potential energy comprises elastic potential energy.

155. The combination of claim 152 in which the potential energy comprises chemical potential energy.

156. The combination of claim 152 in which the potential energy comprises electric potential energy.

157. The combination of claim 156 in which the electric potential energy comprises electrostatic potential energy.

158. The combination of claim 156 in which the electric potential energy comprises electrodynamic potential energy.

159. The combination of claim 156 in which the electric potential energy comprises nuclear potential energy.

160. The combination of claim 152 in which the potential energy comprises thermal potential energy.

161. The combination of claim 152 in which the potential energy comprises rest mass energy.

162. The combination of claim 1 in which the directional effect comprises doing thermodynamic work.

163. The combination of claim 162 in which doing thermodynamic work comprises altering the enthalpy of a system.

164. The combination of claim 162 in which doing thermodynamic work comprises altering the entropy of a system.

165. The combination of claim 162 in which doing thermodynamic work comprises doing pressure-volume work.

166. The combination of claim 1 in which the directional effect comprises doing organizational work.

167. The combination of claim 166 in which doing organizational work comprises altering the order of a system.

168. The combination of claim 166 in which doing organizational work comprises altering the complexity of a system.

169. The combination of claim 166 in which doing organizational work comprises altering the pattern of a system.

170. The combination of claim 166 in which doing organizational work comprises altering the structure of a system.

171. The combination of claim 166 in which doing organizational work comprises altering the emergent property of a system.

172. The combination of claim 166 in which doing organizational work comprises altering the behavior of a system.

173. The combination of claim 1 in which an input modulates a potential energy surface of the system.

174. The combination of claim 173 in which the potential energy surface is modulated vertically.

175. The combination of claim 173 in which the potential energy surface is modulated laterally.

176. The combination of claim 1 in which one or more inputs modulate the potential energy surface of a transition state in the system both vertically and laterally.

177. The combination of claim 173 in which a transition state of the potential energy surface is modulated.

178. The combination of claim 173 in which the input directly interacts with the reactants.

179. The combination of claim 173 in which the input modulates a property of the environment of the reactants.

180. The combination of claim 179 in which the environment comprises an active pocket of an enzyme.

181. The combination of claim 179 in which the environment comprises a supramolecular structure.

182. The combination of claim 181 in which the supramolecular structure comprises an aptamer.

183. The combination of claim 181 in which the supramolecular structure comprises a zeolite.

184. The combination of claim 181 in which the supramolecular structure comprises a polymer.

185. The combination of claim 181 in which the supramolecular structure comprises a carbon nanotube.

186. The combination of claim 173 in which the system comprises an influence mediator.

187. The combination of claim 186 in which the influence mediator comprises a charged bead or a magnetic bead.

188. The combination of claim 186 in which the influence mediator comprises a linker.

189. The combination of claim 173 in which the system is designed using ab initio simulations, catalytic antibodies and/or in vitro evolution.

190. The combination of claim 173 in which the system comprises a modification to enhance an input.

191. The combination of claim 190 in which the modification comprises attaching an enzyme or a supramolecule to a surface of a reaction chamber with an electrical double layer formed at that surface.

192. The combination of claim 190 in which the modification comprises coating a surface of a reaction chamber with a flexible substrate.

193. An apparatus comprising
a site for a reaction, and
a device interacting with a system that has a perturbation, in such perturbed system a non-directional input is applied to a first variable of the system, and based on an asymmetry of the perturbed system, achieving a directional effect in a second variable of the system, the first and second variables comprising a conjugate pair of variables, and in which at least one of the following pertains: the interaction occurs other than by an apparatus and other than in a way that actually achieves the directional effect, or the conjugate pair is other than position and momentum, or the input or the asymmetry is in a dimension other than spatial coordinates, or the directional effect is other than one-dimensional translational motion and other than one-dimensional rotary motion.

194. The apparatus of claim 193 in which the reaction is a chemical reaction.

195. The apparatus of claim 193 in which the reaction is a biochemical reaction.

196. The apparatus of claim 193 in which the device comprises one or more controlled inputs.

197. The apparatus of claim 193 in which an input comprises a controlled voltage.

198. The apparatus of claim 193 in which an input comprises a controlled mechanical force.

199. The apparatus of claim 193 in which an input comprises a controlled temperature.

200. The apparatus of claim 193 in which an input comprises a controlled pressure.

201. The apparatus of claim 193 in which the device comprises a surface.

202. The combination of claim 1 in which the directional effect comprises converting a type of non-chemical energy into another type of non-chemical energy.

203. The combination of claim 202 in which electrical energy is converted into rotary power or mechanical work.

204. The combination of claim 202 in which rotary power or mechanical work is converted into electrical energy.

205. The combination of claim 1 in which the directional effect comprises converting a type of chemical energy into a type of non-chemical energy.

206. The combination of claim 205 in which a chemical fuel energy is converted into a non-chemical energy.

207. The combination of claim 205 in which a chemical energy is converted into electrical energy.

208. The combination of claim 205 in which a chemical energy is converted into rotary power or mechanical work.

209. The combination of claim 1 in which the directional effect comprises converting a type of non-chemical energy into a chemical energy.

210. The combination of claim 209 in which electrical energy is converted into a chemical energy.

211. The combination of claim 209 in which a non-chemical energy is converted into a high energy density chemical fuel.

212. The combination of claim 211 in which the high energy density chemical fuel comprises methane.

213. The combination of claim 211 in which the high energy density chemical fuel comprises ethane.

214. The combination of claim 211 in which the high energy density chemical fuel comprises hydrogen.

215. The combination of claim 209 in which a non-chemical energy is converted into a biofuel.

216. The combination of claim 215 in which the biofuel comprises methanol.

217. The combination of claim 215 in which the biofuel comprises ethanol.

218. The combination of claim 209 in which a non-chemical energy drives a chemical fuel process.

219. The combination of claim 218 in which the chemical fuel process comprises gasoline cracking.

220. The combination of claim 218 in which the chemical fuel process comprises gasoline synthesis.

221. The combination of claim 1 in which the directional effect comprises converting a type of chemical energy into another type of chemical energy.

222. The combination of claim 221 in which the conversion reaction comprises CO₂ reduction.

223. The combination of claim 221 in which the conversion reaction comprises glucose to fructose conversion.

224. The combination of claim 221 in which the conversion reaction comprises ethylene production.

225. The combination of claim 1 in which the directional effect comprises manipulating a chemical reaction.

226. The combination of claim 225 in which the chemical reaction is an intermediate chemical reaction.

227. The combination of claim 225 in which the chemical reaction is a biochemical reaction.

228. The combination of claim 225 in which the manipulation comprises controlling a direction of the reaction.

229. The combination of claim 225 in which the manipulation comprises altering a final substrate and/or a final product concentration or the ratio of the two concentrations.

230. The combination of claim 225 in which the manipulation comprises doing work on the system that the system would otherwise not do, including against or along other influences and/or gradients.

231. The combination of claim 225 in which the manipulation comprises catalyzing the reaction.

232. The combination of claim 225 in which the manipulation comprises specific enhancement and/or suppression of reactions and/or chemical paths.

233. The combination of claim 225 in which the manipulation comprises increasing, decreasing, or reversing a spontaneity of the reaction.

234. The combination of claim 225 in which the manipulation comprises changing a probability of a specific path and/or product, relative to another alternative path or product, to change a yield of the specific path and/or product.

235. The combination of claim 225 in which a result of the method comprises new mixtures and/or products.

236. A new mixture of product produced by applying the method of combination of claim 225.

237. The combination of claim 1 in which the system is used in chemical manufacturing.

238. The combination of claim 1 in which the system is used in industrial processing.

239. The combination of claim 1 in which the system is used in catalysis.

240. The combination of claim 1 in which the system is used in chemical fuel production.

241. The combination of claim 1 in which the system is used in electricity generation.

242. The combination of claim 1 in which the system is used in rotary power or mechanical work generation.

243. The combination of claim 1 in which the system is used in energy storage.

244. The combination of claim 1 in which the system is used in reduction of undesired chemicals.

245. The combination of claim 244 in which the undesired chemical comprise greenhouse gases.

246. The combination of claim 1 in which the directional effect comprises altering the negentropy of a system.

247. The combination of claim 246 in which the system comprises a self-organizing system.

248. The combination of claim 247 in which the self-organizing system comprises a protein.

249. The combination of claim 247 in which the self-organizing system comprises a self-assembling molecule.

250. The combination of claim 246 in which the system comprises a process.

251. The combination of claim 250 in which the process comprises cell signaling.

252. The combination of claim 250 in which the process comprises homeostasis.

253. The combination of claim 250 in which the process comprises a developmental stage of a living organism.

254. The combination of claim 1 in which the system is used in basic life science research.

255. The combination of claim 1 in which the system is used in medicine.

256. The combination of claim 1 in which the system is used in a synthetic life process or a product.

257. The combination of claim 1 in which the directional effect comprises transporting an object.

258. The combination of claim 257 in which the transportation is against an opposing force and/or gradient.

259. The combination of claim 257 in which the object comprises a micro-object.

260. The combination of claim 257 in which the object comprises an ion.

261. The combination of claim 257 in which the object comprises a molecule.

262. The combination of claim 257 in which the object comprises a biomolecule.

263. The combination of claim 257 in which the object comprises a biological cell.

264. The combination of claim 257 in which the object comprises a macro-object.

265. The combination of claim 257 in which the object comprises a transportation vehicle.

266. The combination of claim 1 in which the system is used in mechanics.

267. The combination of claim 1 in which the system is used in biological transportation.

268. The combination of claim 1 in which the system is used in chemical transportation.

269. The combination of claim 1 in which the system is used in vehicular transportation.

270. The combination of claim 1 in which the directional effect comprises altering a property of an object and/or a process.

271. The combination of claim 270 in which the property comprises structure.

272. The combination of claim 270 in which the property comprises complexity.

273. The combination of claim 270 in which the property comprises strength.

274. The combination of claim 270 in which the property comprises elasticity.

275. The combination of claim 270 in which the property comprises weight.

276. The combination of claim 1 in which the system is used in material science.

277. The combination of claim 1 in which the system is used in manufacturing.

278. The combination of claim 1 in which the directional effect comprises altering the electromagnetic property of an object and/or a process.

279. The combination of claim 1 in which the system is used in electronics.

280. The combination of claim 1 in which the system is used in communications.

281. The combination of claim 1 in which the system comprises a pump that is driven by the dynamics of the system.

282. The combination of claim 281 in which the pump alters the concentration of an object.

283. The combination of claim 206 in which the pump alters the transfer speed of an object.

284. A combination comprising
interaction with a system that has a perturbation,
in such perturbed system an input is applied to a first variable of the system, and
based on an asymmetry of the perturbed system, achieving a directional effect in a second variable of the system,
the first and second variables comprising a conjugate pair of variables.

285. A combination comprising
interaction with a system that has a perturbation,
in such perturbed system an input is applied to a first variable of the system, and
based on an asymmetry of the perturbed system, achieving a directional effect in a second variable of the system,
the first and second variables comprising a conjugate pair of variables other than position and momentum.

286. A combination comprising
non-physical interaction with a system that has a perturbation,
in such perturbed system an input is applied to a first variable of the system, and
based on an asymmetry of the perturbed system, indirectly achieving a directional effect in a second variable of the system,
the first and second variables comprising a conjugate pair of variables.

287. A combination comprising
interaction with a system that has a perturbation,
in such perturbed system an input is applied to a first variable of the system, and
based on an asymmetry of the perturbed system, achieving a directional effect in a second variable of the system,
the first and second variables comprising a conjugate pair of variables,
the input or the asymmetry is in a dimension other than spatial coordinates.

288. The combination of claim 1 wherein the combination comprises one or more actions or steps in a method, one or more elements in an apparatus, one or more parts in a combination of matter, or sub-combinations thereof.

289. In combination, the features of claims 1, 2, 42, 45, 49, 51, 54, 56, 57, 64, 71, 75, 78, 79, 81, 83, 84, 85, 87, 92, 93, 95, 97, 98, 100, 106, 108, 110, 112, 114, 118, 121, 126, 127, 128, 129, 130, 131, 132, 133, 135, 136, 137, 142, 143, 145, 152, 155, 162, 163, 164, 173, 176, 177, 179, 180, 209, 210, 211, 214, 225, 229, 230, 233, and 240.
